(19) United States
(12) Patent Application Publication
(10) Pub. No.: US 2020/0148785 A1
(43) Pub. Date: May 14, 2020

(54) PD-L1 AND TA-MUC1 ANTIBODIES

(71) Applicant: Glycotope (GmbH), Berlin (DE)

(72) Inventors: Patrik Kehler, Berlin (DE); Steffen Goletz, Berlin (DE); Antje Danielczyk, Panketal (DE); Johanna Ruehmann, Berlin (DE); Christoph Goletz, Berlin (DE)

(21) Appl. No.: 16/499,058

(22) PCT Filed: Mar. 28, 2018
(86) PCT No.: PCT/EP2018/057844

§ 371 Date: Sep. 27, 2019

(30) Foreign Application Priority Data
Mar. 29, 2017 (LU) LU100150
May 15, 2017 (EP) 17171013.0

(51) Int. Cl. C07K 16/30; C07K 16/28; A61P 35/00

(52) U.S. Cl. CPC C07K 16/3092; C07K 16/2827; A61P 35/00; C07K 2317/76; C07K 2317/41; C07K 2317/72; C07K 2317/732; C07K 2317/31

(57) ABSTRACT

The present invention relates to an antibody which effects enhanced T cell activation in comparison to a reference antibody being glycosylated including more than 80% core-fucosylation and wherein T cell activation is effected by an antibody characterized by enhanced binding to Fc#RIIIa. Said antibody is glycosylated, but essentially lacks core-fucosylation.

### FIELD OF THE INVENTION

The present invention relates to an antibody which effects enhanced T cell activation in comparison to a reference antibody being glycosylated including more than 80% core-fucosylation. Further, the antibody effects enhanced T cell activation in comparison to a reference antibody being non-glycosylated and wherein T cell activation is effected by an antibody characterized by an enhanced binding to FcγRIIIa. Said antibody is glycosylated, but essentially lacks core-fucosylation.

### BACKGROUND

### Immune Checkpoint Protein Blockade

The Programmed death-ligand 1 (PD-L1) also known as cluster of differentiation 274 (CD274) or B7 homolog 1 (B7-H1) is a protein that in humans is encoded by the CD274 gene and refers to an immune checkpoint protein.

It is constitutively expressed on immune cells such as T and B cells, dendritic cells (DCs), macrophages, mesenchymal stem cells and bone marrow-derived mast cells (Yamazaki et al., 2002, J. Immunol. 169: 5538-45). According to Keir et al. (2008), Annu. Rev. Immunol. 26: 677-704, PD-L1 can also be expressed on a wide range of non-hematopoietic cells such as cornea, lung, vascular epithelium, liver non-parenchymal cells, mesenchymal stem cells, pancreatic islets, placental synctiotrophoblasts, keratinocytes, etc. Further, upregulation of PD-L1 is achieved on a number of cell types after activation of said cells. A major role was assigned to PD-L1 in suppressing the immune system during tissue autoimmune disease, allografts, and other disease states.

PD-L1 binds to the programmed death-1 receptor (PD-1) (CD279), which provides an important negative co-stimulatory signal regulating T cell activation. PD-1 can be expressed on all kinds of immune cells such as T cells, B cells, natural killer T cells, activated monocytes and DCs. PD-1 is expressed by activated, but not by unstimulated human CD4⁺ and CD8⁺ T cells, B cells and myeloid cells. Additionally, besides binding to PD-L1, PD-1 also binds to its ligand binding partner PD-L2 (B7-DC, CD273). PD-1 is related to CD28 and CTLA-4, but lacks the membrane proximal cysteine that allows homo-dimerization.

In general, the binding of PD-L1 to PD-1 transmits an inhibitory signal which reduces the proliferation of CD8⁺ T cells.

PD-L1 is also considered as a binding partner for B7.1 (CD80) (Butte et al., 2007, Immunity 27: 111-22). Chemical crosslinking studies suggest that PD-L1 and B7.1 can interact through their IgV-like domains. Moreover, B7.1-PD-L1 interactions can induce an inhibitory signal into T cells.

When T cells lacking all known receptors for PD-L1 (i.e., no PD-1 and B7.1), T cell proliferation is no longer impaired. In other words an impairment of the engagement of PD-L1 with its receptor PD-1 on T cells leads to T cell receptor-mediated activation of IL-2 production and T cell proliferation. Thus, PD-L1 plays a specific role in inhibiting T cells either through B7.1 or PD-1.

Cancer cells may also upregulate PD-L1 as well, thus allowing cancers to evade the host immune system. PD-L1 is expressed on a variety of different cancer types including, but not limited to carcinomas, sarcomas, lymphomas and leukemia, germ cell tumors and blastomas. Loss or inhibition of phosphatase and tensin homolog (PTEN), a cellular phosphatase that modified phosphatidylinositol 3-kinase (PI3K) and Akt signaling, increased post-transcriptional PD-L1 expression in cancers (Parsa et al., 2007, Nat. Med. 13: 84-88).

Particularly, enhancement of T cell immunity for cancer treatment (e.g. tumor immunity) and acute or chronic infection is strongly associated with the inhibition of PD-L1 signaling.

As a therapeutic treatment for cancer, it is thus common to apply specific antibodies targeting the PD-L1/PD-1 axis (f.e. anti-PD-L1 or anti-PD-1) or PD-L1/CD80 interaction being able to target cancer cells in therapy, which is a highly promising and clinically proven concept.

### ADCC and ADCP Activity

The ability to mediate cellular cytotoxic effector functions such as Antibody-dependent cell cytotoxicity (ADCC) and Antibody-dependent cell-mediated phagocytosis (ADCP) is a promising means to enable the enhancement of the antitumor potency of antibodies.

In general, for IgG class antibodies ADCC and ADCP are mediated by engaging of the F_{c} region with specific so called Fc gamma receptors (FcγRs). There are three classes of receptors in humans: the FcγRI (CD64), FcγRII (CD32) with its isoforms FcγRIIa, FcγRIIb and FcγRIIc, and FcγRIII (CD16) with its isoforms FcγRIIa and FcγRIIb. The same region on IgG Fc is bound by all FcγRs, only differing in their affinities with FcγRI having a high affinity and FcγRII and FcγRIII having a low affinity. Therefore, an antibody with an optimized FcγR affinity may result in a better functionality resulting in better cellular antitumor effects in therapy.

ADCC is a mechanism whereby the antibody binds with its F_{ab} region to a target cell antigen and recruits effector cells by binding of its F_{c} part to Fc receptors on their surface of these cells, resulting in the release of cytokines such as IFN-γ and cytotoxic granules containing perforin and granzymes that enter the target cell and promote cell death. It was found that in particular the FcγRIIIa plays the most crucial role in mediating ADCC activity to targeted cancer cells.

It is known from the literature that modifications of the oligosaccharide structure in the F_{c} region (F_{c} N-glycosylation) predominantly influences binding of antibodies to the Fc receptor and are an established approach for enhancing ADCC activity. In general, glycosylation itself and variations in glycoforms are being known to play an important role by affecting biological functions of IgG antibodies.

In general, glycosylated antibodies may comprise two N-linked oligosaccharides at each conserved asparagine 297 (N297), according to EU-nomenclature, in the CH₂ domain. Typically, N-glycans attached to each N297 of the antibody may be of the complex type but also highmannose or hybride type N-glycans may be linked to each N297 of the antibody. The complex type N-glycosylation may be characterized by a mannosyl-chitobiose core (Man3GlcNAc2-Asn) with variations in the presence/absence of bisecting N-acetylglucosamine and core-fucose, which may be α-1.6-linked to the N-acetylglucosamine that is attached to the antibodies. Furthermore, the complex type N-glycosylation may be characterized by antennary N-acetylglucosamine linked to the mannosyl-chitobiose core (Man3GlcNAc2-Asn) with optional extension of the antenna by galactose and sialic acid moieties. Additionally, antennary fucose and/or N-acetylgalactosamine may be part of the extension of the antenna as well.

Since cancer cells upregulate the “tumor-associated mucin 1 epitope TA-MUC1”, ADCC activity commonly plays an important role in cancer therapy through the application of antibodies, targeting TA-MUC1 positive cancer cells.

TA-MUC1 is present on cancer cells but not on normal cells and/or it is only accessible by antibodies in the host's circulation when present on tumor cells but not when present on normal cells. Targeting TA-MUC1 provides specific direction and accumulation into the tumor. Overexpression of TA-MUC1 is often associated with colon, breast, ovarian, lung and pancreatic cancers.

### Enhanced T Cell Activation

The first time T cells encounter their specific antigen in the form of a peptide:MHC complex on the surface of an activated antigen-presenting cell (APC), naive T cells become activated. The most important antigen-presenting cells are the highly specialized dendritic cells (DCs), functioning through ingesting and presenting antigens. Tissue dendritic cells ingest antigen at sites of infection and are activated as part of the innate immune response. They migrate then to local lymphoid tissue and mature into cells that are highly effective at presenting antigen to recirculating T cells. The characterization of these mature dendritic cells is based on surface molecules, known as co-stimulatory molecules that synergize with antigen in the activation of naive T cells into effector T cells.

Depending on the peptide antigens (e.g. intracellular and extracellular) presented by the DCs to T cells, different T cells are being activated. Extracellular peptides are carried to the cell surface by MHC class II molecules and presented to CD4 T cells. Amongst others, two major types of effector T cells, called T_{H}1 and T_{H}2 are differentiated thereof. Intracellular antigens are carried to the cell surface by MHC class I molecules and presented to CD8 T cells. After differentiation into cytotoxic T cells they kill infected target cells, such as cancer cells. (Janeway et al., 2001, “Immunobiology: The Immune System in Health and Disease”, Garland Science, 5th edition). Therefore, in cancer therapy and also in other diseases, T cell activation plays an important role.

The object of the present invention is to provide an improved antibody, which may be used for different therapeutic applications.

### SUMMARY OF THE INVENTION

The present invention provides an antibody, which effects enhanced T cell activation in comparison to an antibody being glycosylated including more than 80% core-fucosylation, wherein the reference antibody is preferably obtainable from CHOdhfr− (ATCC No. CRL-9096). In particular, the present invention may envisage a glycosylated antibody essentially lacking core-fucosylation, which effects enhanced T cell activation in comparison to an antibody being glycosylated including more than 80% core-fucosylation. Preferably, an antibody of the present invention may be from 0% to 80% fucosylated.

An antibody of the present invention may effect enhanced T cell activation also in comparison to a reference antibody being non-glycosylated. Further, said T cell activation of the present invention may be effected by an antibody of the present invention characterized by an enhanced binding to FcγRIIIa.

The invention may also encompass an antibody, wherein said glycosylation is human glycosylation. Additionally, the glycosylation of the reference antibody including more than 80% core-fucosylation may also be human glycosylation.

Additionally, the present invention may contemplate an antibody, wherein said antibody may be obtainable from the cell line NM-H9D8-E6 (DSM ACC 2807), NM-H9D8-E6Q12 (DSM ACC 2856), or a cell or cell line derived therefrom. The antibody of the present invention may also comprise one or more sequence mutations, wherein the binding of said antibody to FcγRIIIa is preferably increased compared to a non-mutated antibody. Further, the present invention may provide an antibody of the present invention, wherein the antibody may comprise one or more sequence mutations selected from S238D, S239D, 1332E, A330L, S298A, E333A, L334A, G236A and L235V according to EU-nomenclature.

The present invention may further contemplate an antibody of the present invention, wherein T cell activation may be accompanied by maturation of dendritic cells and/or expression of co-stimulatory molecules and maturation markers and wherein said T cell activation may be detectable by the expression CD25, CD69 and/or CD137.

The present invention may provide an antibody, wherein said antibody is preferably a PD-L1 antibody. Said PD-L1 antibody of the present invention may be a bifunctional monospecific antibody or a trifunctional bispecific antibody. Being a trifunctional bispecific antibody, said PD-L1 antibody may further bind to a cancer antigen, wherein said cancer antigen is preferably TA-MUC1. Additionally, said PD-L1 antibody of the present invention may comprise an F_{c} region.

The present invention may provide an antibody of the present invention, wherein said antibody is preferably a TA-MUC1 antibody. Said TA-MUC1 antibody may be a bifunctional monospecific antibody or a trifunctional bispecific antibody. Being a trifunctional bispecific antibody, said TA-MUC1 antibody may further bind to an immune checkpoint protein, wherein said immune checkpoint protein is preferably PD-L1. Additionally, said TA-MUC1 antibody of the present invention may comprise an F_{c} region and single chain Fᵥ regions binding to PD-L1. Further, said TA-MUC1 antibody may comprises V_{H} and V_{L} domains binding to TA-MUC1. The single chain Fᵥ regions of said TA-MUC1 antibody may be coupled to the constant domain of the light chain or to the CH₃ domain of the F_{c} region.

The present invention may provide an antibody of the present invention, a monospecific or bispecific PD-L1 antibody and/or a monospecific or bispecific TA-MUC1 antibody for use in therapy. Further, the present invention may provide an antibody, a monospecific or bispecific PD-L1 antibody and/or a monospecific or bispecific TA-MUC1 antibody for use in a method for activating T-cells. Additionally, the present invention may encompass an antibody of the present invention, wherein the activation of T-cells is preferably for the treatment of cancer disease, inflammatory disease, virus infectious disease and autoimmune disease. In particular, cancer disease may be selected from Melanoma, Carcinoma, Lymphoma, Sarcoma, and Mesothelioma including Lung Cancer, Kidney Cancer, Bladder Cancer, Gastrointestinal Cancer, Skin Cancer, Breast Cancer, Ovarian Cancer, Cervical Cancer, and Prostate Cancer. Additionally, inflammatory disease may be selected from Inflammatory Bowel Disease (IBD), Pelvic Inflammatory Disease (PID), Ischemic Stroke (IS), Alzheimer's Disease, Asthma, Pemphigus Vulgaris, Dermatitis/Eczema. Virus infectious disease may be selected from Human Immunodeficiency Virus (HIV), Herpes Simplex Virus (HSV), Epstein Barr Virus (EBV), Influenza Virus, Lymphocytic Choriomeningitis Virus (LCMV), Hepatitis B Virus (HBV), Hepatitis C Virus (HCV). Further, autoimmune disease may be selected from Diabetes Mellitus (DM), Type I, Multiple Sclerosis (MS), Systemic Lupus Erythematosus (SLE), Rheumatoid Arthritis (RA), Vitiligo, Psoriasis and Psoriatic Arthritis, Atopic Dermatitis (AD), Scleroderma, Sarcoidosis, Primary Biliary Cirrhosis, Guillain-Barre Syndrome, Graves' Disease, Celiac Disease, Auto-immune Hepatitis, Ankylosing Spondylitis (AS).

### BRIEF DESCRIPTION OF THE FIGURES

FIG. 1: Measuring core fucosylation.

The monospecific PDL-GEX Fuc− and bispecific PM-PDL-GEX Fuc− have reduced core fucosylation compared to the monospecific PDL-GEX H9D8 and bispecific PM-PDL-GEX H9D8. The relative molar amounts of core fucosylated N-glycans of monospecific antibodies PDL-GEX H9D8 and PDL-GEX Fuc− and of bispecific antibodies PM-PDL-GEX H9D8 and PM-PDL-GEX Fuc− are illustrated herein. The monospecific PDL-GEX H9D8 and the bispecific PM-PDL-GEX H9D8 contain 92% and 91% of core fucosylated N-glycans, respectively, and are therefore referred as normal-fucosylated. The monospecific PDL-GEX Fuc− and the bispecific PM-PDL-GEX Fuc− contain only low percentages of core fucosylated N-glycans, preferably 4% for PDL-GEX Fuc− and 1% for PM-PDL-GEX Fuc−, and are therefore referred as fucose-reduced. This is described in Example 1.

FIG. 2: Blocking capacity of fucose-reduced and normal fucosylated antibodies.

A fucose-reduced anti-PD-L1 hIgG1 and a fucose-reduced bispecific anti-PD-L1/TA-MUC1 hIgG1 show comparable blocking capacity compared to their normal-fucosylated counterparts: A) Concentration-dependent blocking of PD-1 binding was detected for all four variants and no difference in the PD-L1/PD-1 blocking ELISA between normal- and fucose-reduced anti-PD-L1 hIgG1 (PDL-GEX-H9D8 and PDL-GEX-Fuc−), and normal- and fucose-reduced bispecific anti-PD-L1/TA-MUC1 hIgG1 (PM-PDL-GEX-H9D8 and PM-PDL-GEX-Fuc−), respectively, was detected. The slight reduction in inhibition of the bispecific anti-PD-L1/TA-MUC1 hIgG1 is presumably due to transformation of the anti-PD-L1 hIgG1 into an anti-PD-L1 scFᵥ format. B) All four variants (PDL-GEX-H9D8, PDL-GEX-Fuc−, PM-PDL-GEX-H9D8 and PM-PDL-GEX-Fuc) tested show effective inhibition of the interaction between PD-L1 and CD80 and no obvious difference between the glycosylation variants (fucose reduced- vs. normal-fucosylated) was detected. This is described in Example 2.

FIG. 3: Binding capacity to TA-MUC1.

Both, the fucose-reduced and the normal-fucosylated bispecific anti-PD-L1/TA-MUC1 hIgG1 (PM-PDL-GEX Fuc− and PM-PDL-GEX-H9D8) show comparable binding to TA-MUC1. As expected, the monospecific anti-PD-L1 (PDL-GEX H9D8) shows no binding to the breast cancer cell line ZR-75-1. This is described in Example 3.

FIG. 4: Binding capacity to FcyRIIIa.

The fucose-reduced variants of an anti-PD-L1 hIgG1 and a bispecific anti-PD-L1/TA-MUC1 hIgG1 show increased binding to FcyRIIIa compared to the normal-fucosylated variants: The comparison of the different fucosylation variants of anti-PD-L1 hIgG1 and the bispecific anti-PD-L1/TA-MUC1 hIgG1 is illustrated herein. The fucose-reduced anti-PD-L1 (PDL-GEX Fuc−) has a decreased EC50 value compared to the normal-fucosylated anti-PD-L1 hIgG1 (PDL-GEX H9D8) demonstrating ˜5-fold enhanced binding to FcγRIIIa of the fucose-reduced variant compared to the normal-fucosylated variant.

The bispecific fucose-reduced and normal-fucosylated anti-PD-L1/TA-MUC1 hIgG1 were not compared in the same experiment, but they were quantitatively compared by calculation of a relative potency compared to a normal-fucosylated reference antibody (EC50 of reference antibody divided by EC50 of test antibody). For the bispecific normal-fucosylated anti-PD-L1/TA-MUC1 hIgG1 (PM-PDL-GEX H9D8) a relative potency of 1.9 was determined. In contrast, the relative potency of the bispecific fucose-reduced anti-PD-L1/TA-MUC-1 hIgG1 (PM-PDL-GEX Fuc−) was determined as 10.4. From that, the binding to FcγRIIIa is also enhanced by ˜5-fold for the fucose-reduced variant (PM-PDL-GEX Fuc−) compared to the normal-fucosylated counterpart (PM-PDL-GEX H9D8). This is described in Example 4.

FIG. 5: Measuring ADCC activity against TA-MUC⁺ and PD-L1⁺ tumor cells.

A fucose-reduced anti-PD-L1 hIgG1 and a fucose-reduced bispecific anti-PD-L1/TA-MUC1 hIgG1 show increased killing of TA-MUC+ and PD-L1+ tumor cells compared to their normal-fucosylated counterparts: A) Due to increased binding to FcγRIIIa, the fucose-reduced bispecific anti-PD-L1/TA-MUC1 hIgG1 (PM-PDL-GEX Fuc−) shows strongly enhanced ADCC activity compared to the normal-fucosylated bispecific anti-PD-L1/TA-MUC1 hIgG1 (PM-PDL-GEX-H9D8) against the breast cancer cell line ZR-75-1 which expresses high levels of TA-MUC1 and only marginal levels of PD-L1. The monospecific anti-PD-L1 antibodies (PDL-GEX Fuc− and PDL-GEX H9D8) show no ADCC as expected, since the target cells express minimal/no PD-L1. The prostate carcinoma cell line DU-145 strongly expresses PD-L1 (B) and has moderate TA-MUC1 expression (C). D) The fucose-reduced anti-PD-L1 (PDL-GEX Fuc−) and the fucose-reduced bispecific anti-PD-L1/TA-MUC1 hIgG1 (PM-PDL-GEX Fuc−) mediate strongly enhanced ADCC against PD-L1 positive tumor cells compared to their normal-fucosylated counterparts. The slight reduction in the ADCC effect of the bispecific formats compared to their corresponding monospecific anti-PD-L1 hIgG1 is presumably due to transformation of the anti-PD-L1 hIgG1 into an anti-PD-L1 scFᵥ format. This is described in Example 5.

FIG. 6: Measuring ADCC activity against PD-L1⁺ PBMCs.

A fucose-reduced anti-PD-L1 hIgG1 and a fucose-reduced bispecific anti-PD-L1/TA-MUC1 hIgG1 show no ADCC effect against PD-L1+ PBMCs: Surprisingly, no ADCC effect mediated by fucose-reduced anti-PD-L1 (PDL-GEX-Fuc−) and fucose-reduced bispecific anti-PD-L1/TA-MUC1 (PM-PDL-GEX-Fuc−) against B cells (A) and monocytes (B) was detected. In contrast, the positive control Gazyvaro® induces killing of both, primary B cells and Daudi cells. For monocytes, staurosporine as a positive control induces killing of monocytes and THP-1 control cells. This is described in Example 6.

FIG. 7: Measuring PD-1/PD-L1 blockade.

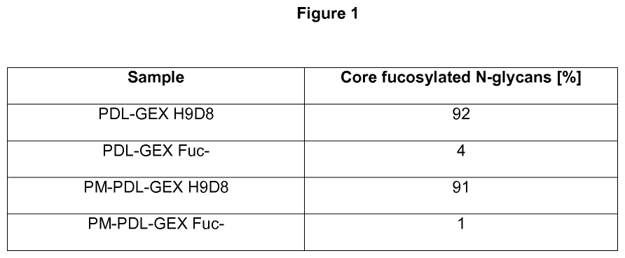

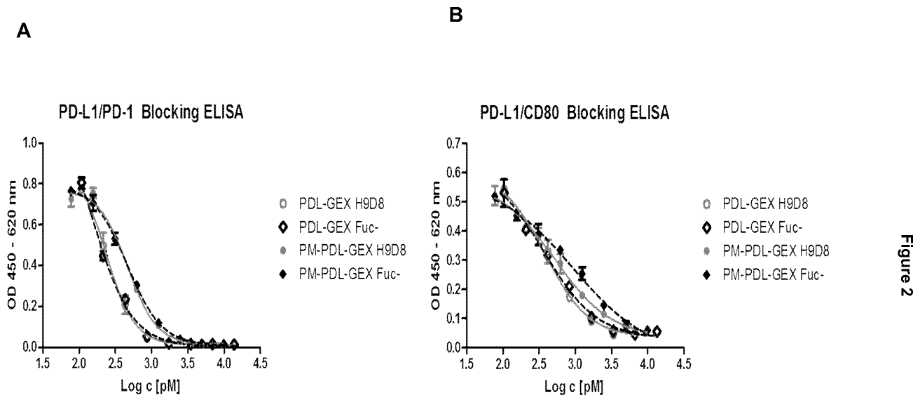

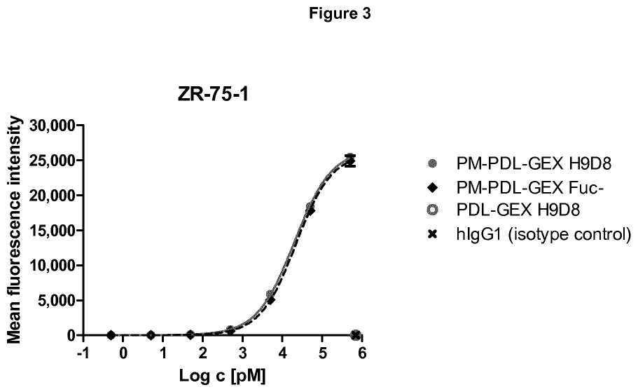

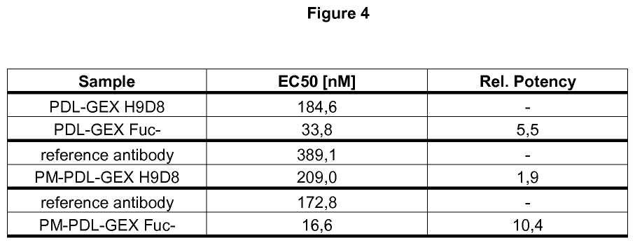

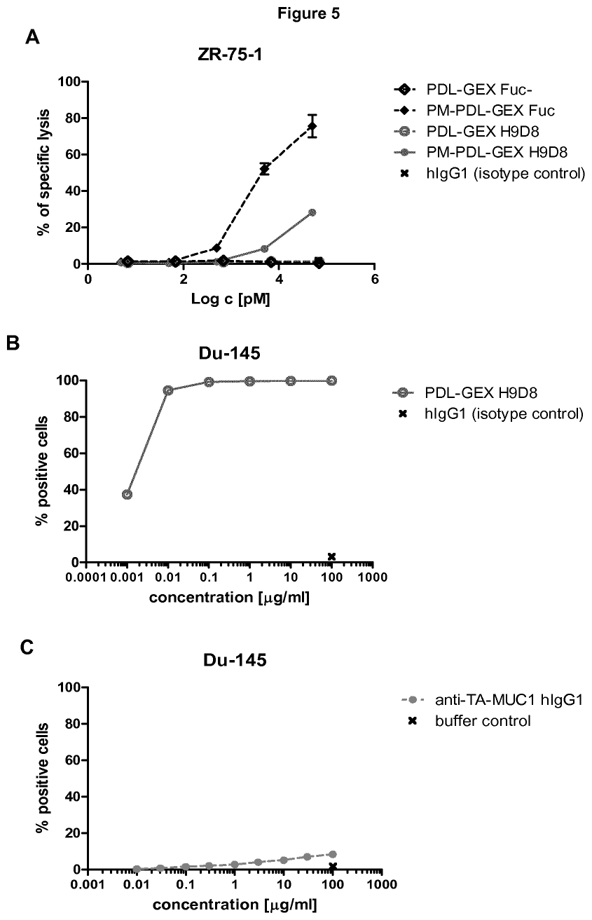

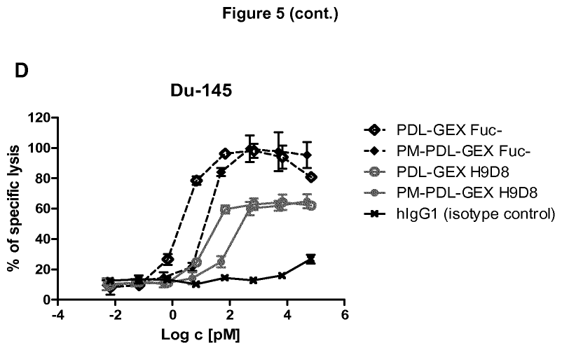

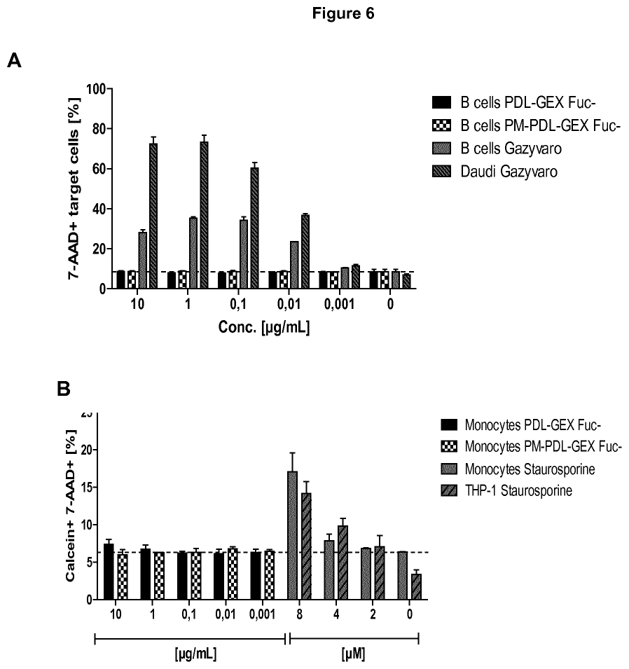

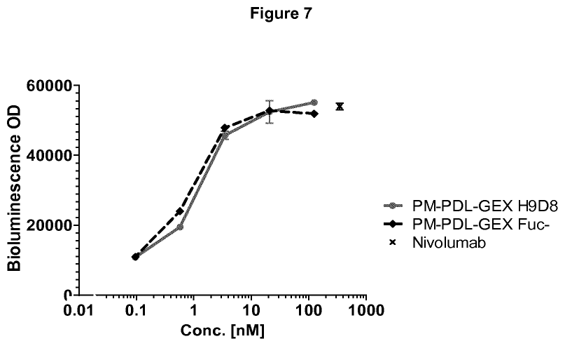

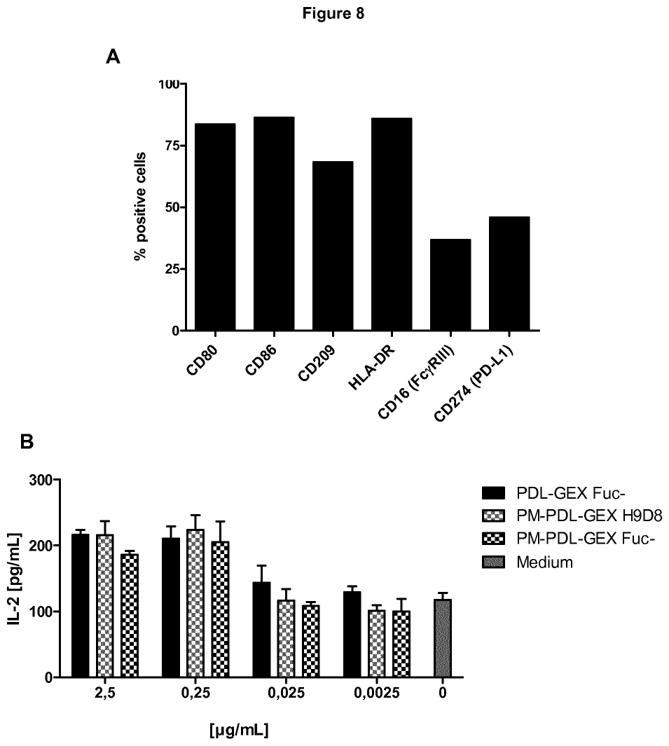

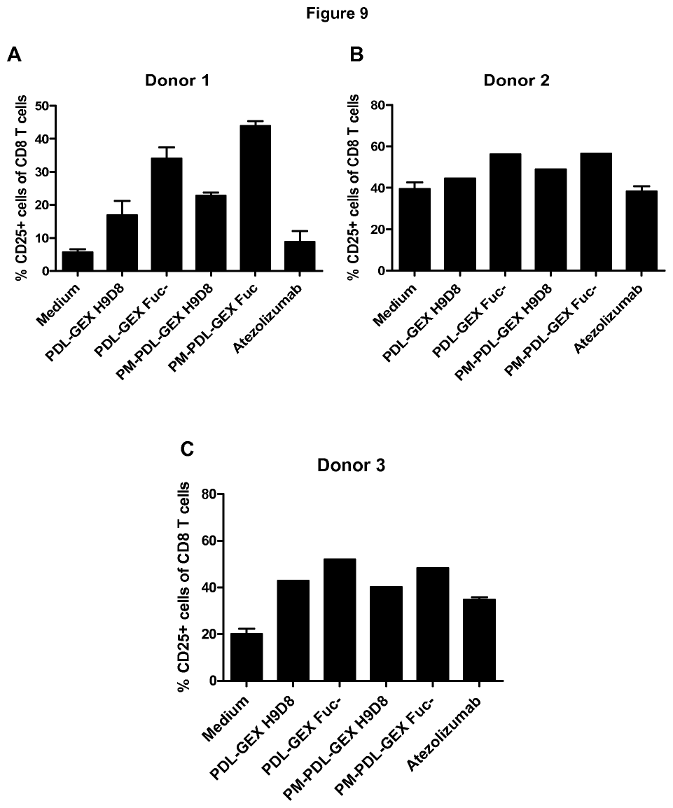

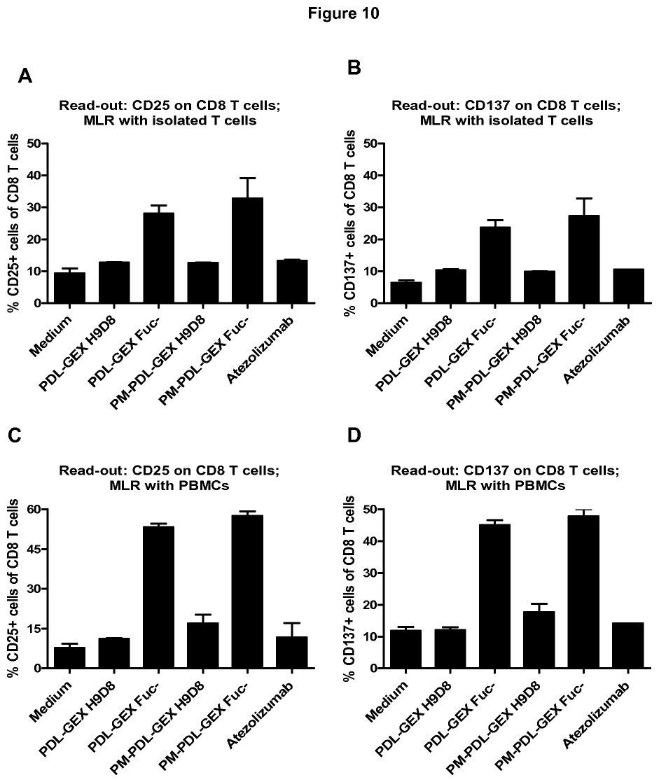

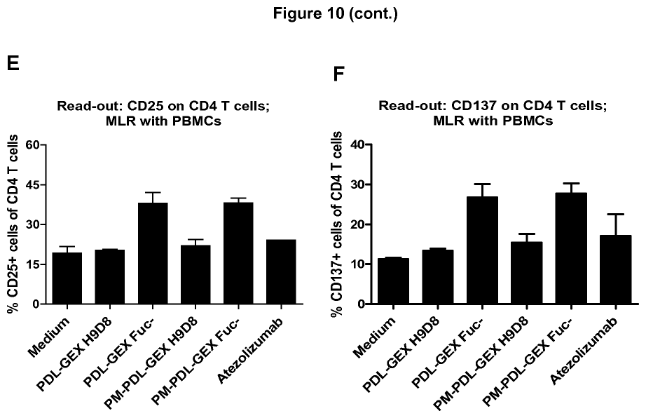

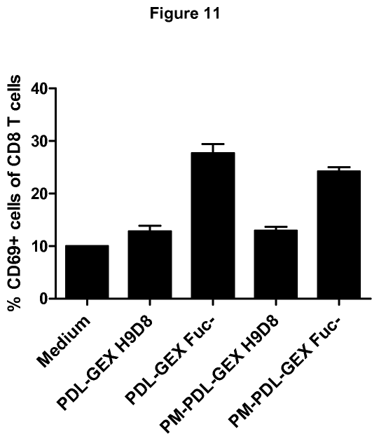

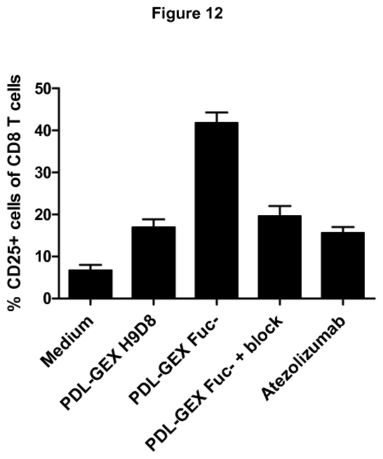

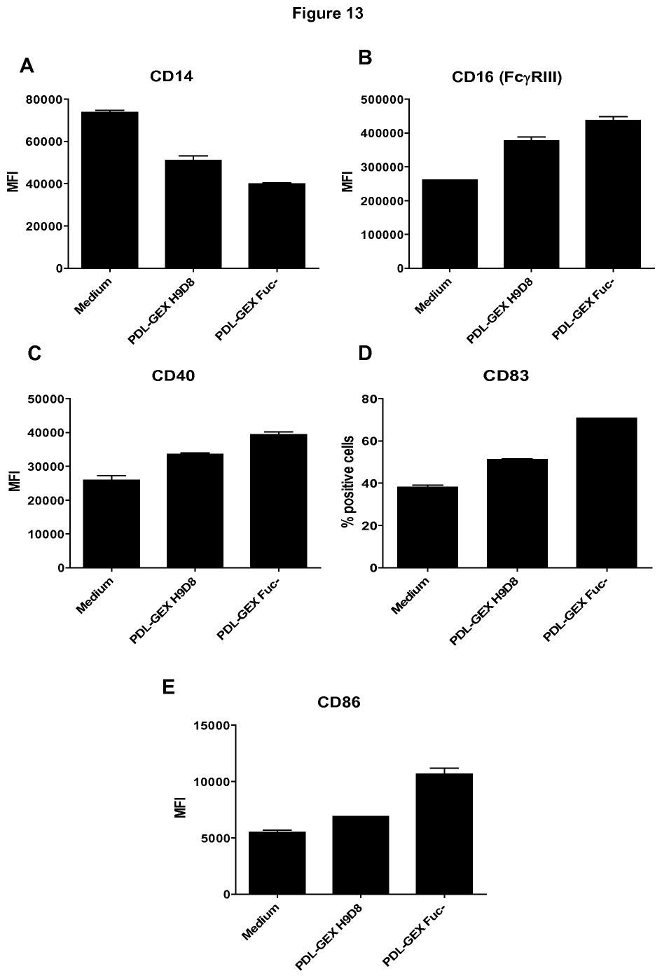

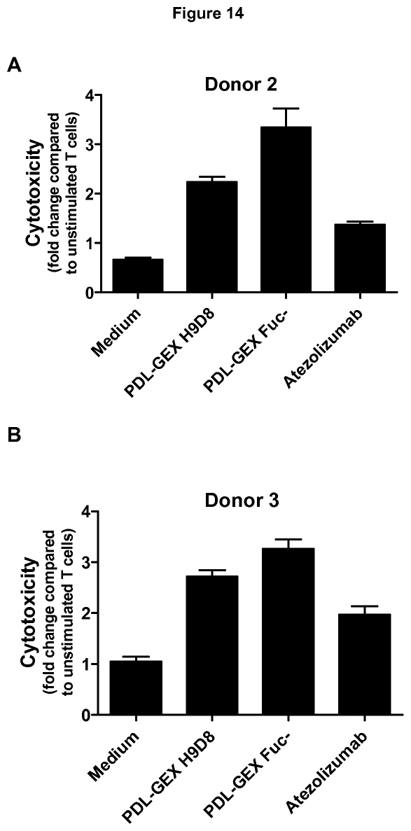

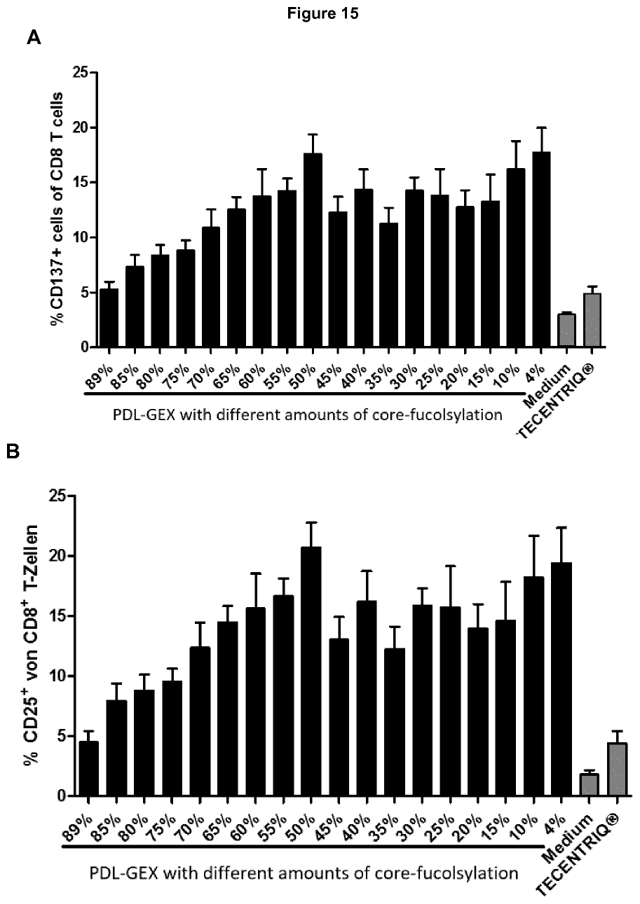

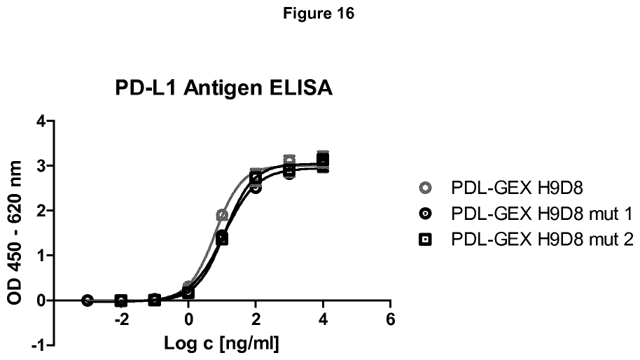

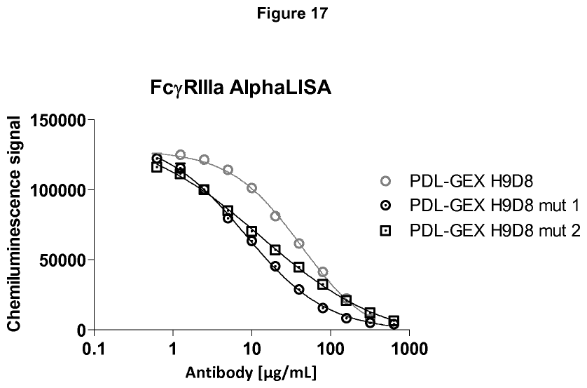

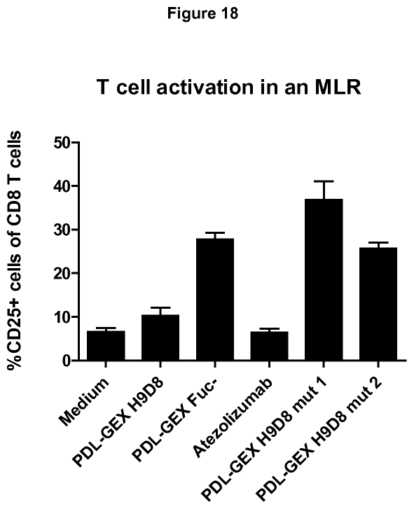

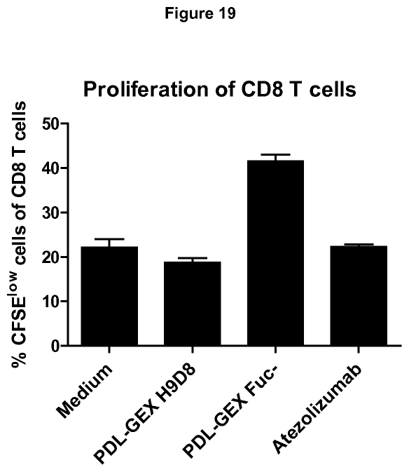

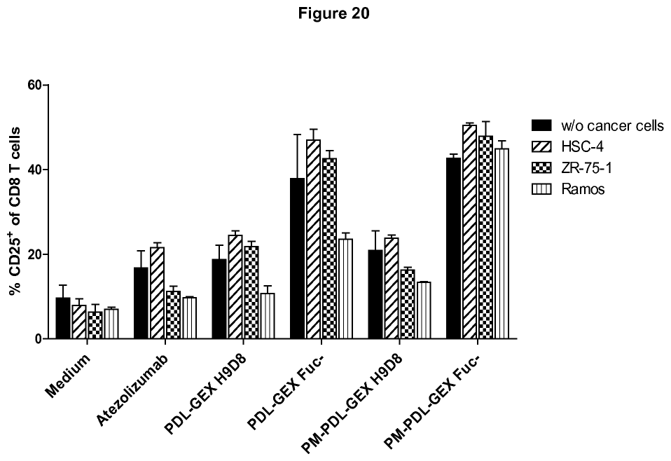

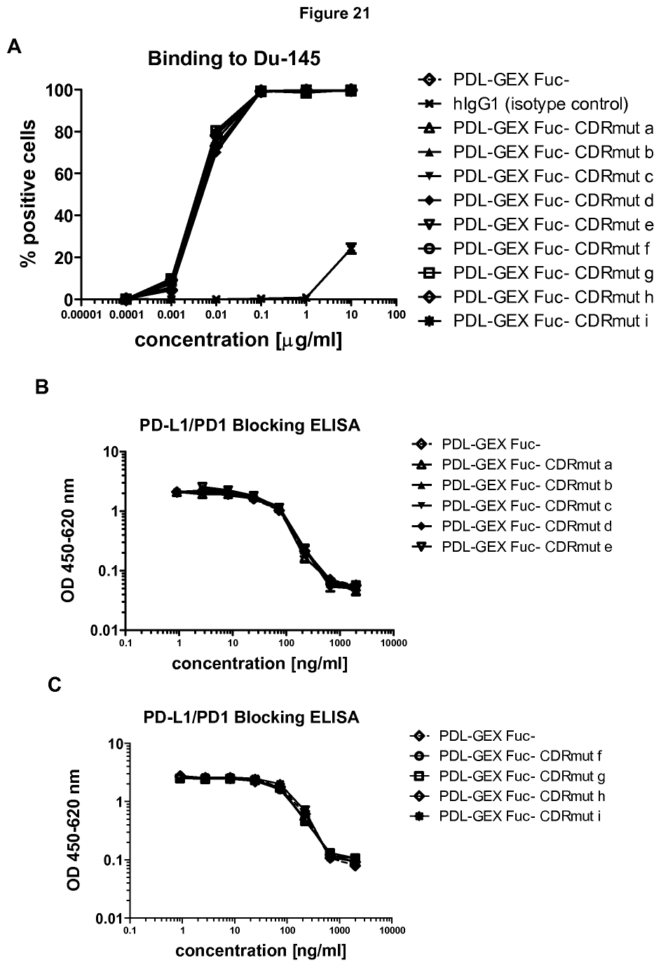

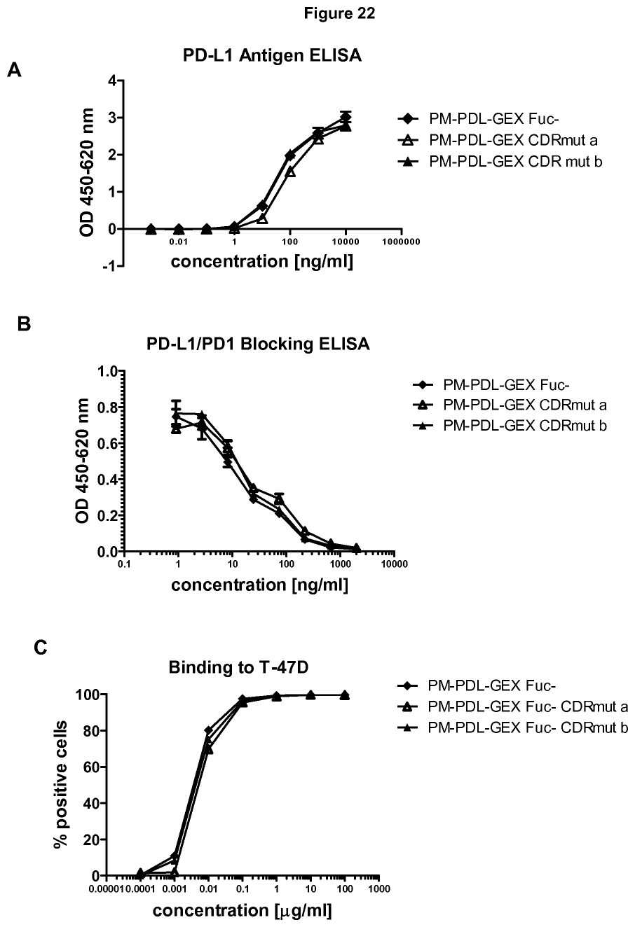

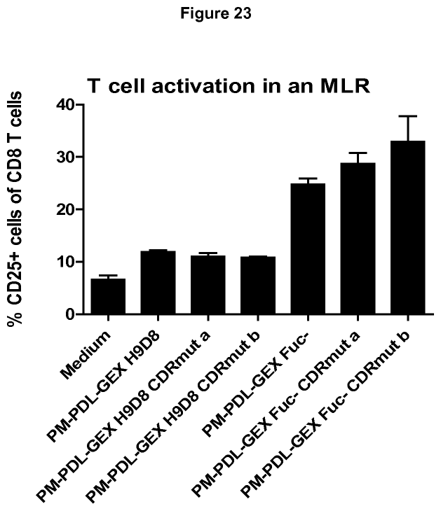

A fucose-reduced and a normal-fucosylated bispecific anti-PD-L1/TA-MUC1 hIgG1 show comparable results in a cell based PD-1/PD-L1 blockade bioassay. Comparable dose-dependent release of the PD-1/PD-L1 break was detected for both, the fucose-reduced (PM-PDL-GEX Fuc−) and normal-fucosylated (PM-PDL-GEX H9D8) bispecific anti-PD-L1/TA-MUC1 hIgG1 in accordance with the PD-L1/PD-1 block ELISA (see FIG. 1). As expected, Nivolumab was effective as the positive control. This is described in Example 7.

FIG. 8: Measuring of IL-2 in MLRs.

A fucose-reduced and a normal-fucosylated bispecific anti-PD-L1/TA-MUC1 hIgG1 and a fucose-reduced anti-PD-L1 hIgG1 induce comparable IL-2 in an allogeneic mixed lymphocyte reaction (MLR). A) A representative experiment analyzing the phenotype of moDCs by flow cytometry. MoDCs expressed the co-stimulatory molecules CD80 and CD86, the DC-marker CD209 and the MHC class II surface receptor HLA-DR. In addition, moDCs were found to express CD16 (FcγRIII) and CD274 (PD-L1). B) No influence of de-fucosylation on IL-2 secretion was detected since the fucose-reduced (PM-PDL-GEX Fuc−) and the normal-fucosylated bispecific anti-PD-L1/TA-MUC1 hIgG1 (PM-PDL-GEX H9D8) and the fucose-reduced anti-PD-L1 hIgG1 (PDL-GEX Fuc−) induced comparable amount of IL-2. This is described in Example 8.

FIG. 9: Measuring T cell activation.

A fucose-reduced anti-PD-L1 hIgG1 and fucose-reduced bispecific anti-PD-L1/TA-MUC1 hIgG1 show increased T cell activation compared to normal-fucosylated counterparts and an anti-PD-L1 antibody with no/weak FcyR-binding capacity. Results obtained with isolated T cells from three different healthy volunteers ((A)=donor 1, (B)=donor 2 and (C)=donor 3) in allogeneic MLRs demonstrate that a fucose-reduced anti-PD-L1 hIgG1 (PDL-GEX Fuc−) and a fucose-reduced bispecific anti-PD-L1/TA-MUC1 hIgG1 (PM-PDL-GEX Fuc−) induce enhanced T cell activation compared to their normal-fucosylated monospecific anti-PD-L1 hIgG1 (PDL-GEX H9D8) and bispecific anti-PD-L1/TA-MUC1 hIgG1 (PM-PDL-GEX H9D8) counterparts, also compared to an anti-PD-L1 antibody with no/weak FcyR-binding capacity (Atezolizumab). This is described in Example 9.

FIG. 10: Measuring T cell activation in a MLR with isolated T cells and total PBMCs.

A fucose-reduced anti-PD-L1 hIgG1 and fucose-reduced bispecific anti-PD-L1/TA-MUC1 hIgG1 show increased T cell activation compared to normal-fucosylated counterparts and an anti-PD-L1 with no/weak FcyR-binding capacity in a MLR with isolated T cells and total PBMCs. Flow cytometric analysis shows that the fucose-reduced monospecific anti-PD-L1 hIgG1 (PDL-GEX Fuc−) and the fucose-reduced bispecific anti-PD-L1/TA-MUC1 hIgG1 (PM-PDL-GEX Fuc−) induce stronger CD8 T cell activation compared to a normal-fucosylated monospecific anti-PD-L1 hIgG1 (PDL-GEX H9D8), to a bispecific anti-PD-L1/TA-MUC1 hIgG1 (PM-PDL-GEX H9D8) and compared to an anti-PD-L1 with no/weak FcyR-binding capacity (Atezolizumab) measured by expression of CD25 and CD137 on CD3⁺CD8⁺ cells using either T cells (A, B) or PBMCs (C, D) as responder cells in the MLR. Cultivation of moDCs with PBMCs additionally leads to increased CD4 T cell activation (CD3⁺CD8⁻ cells ergo CD4 T cells) due to the fucose-reduced monospecific PDL-GEX Fuc− and the fucose-reduced bispecific PM-PDL-GEX Fuc− measured by expression of CD25 (E) and CD137 (F), which was not observed earlier in MLRs using isolated T cells. This is described in Example 10.

FIG. 11: Measuring CD69 expression on T cells.

A fucose-reduced anti-PD-L1 hIgG1 and fucose-reduced bispecific anti-PD-L1/TA-MUC1 hIgG1 also increase CD69 expression on T cells. Flow cytometric analysis shows that the fucose-reduced monospecific anti-PD-L1 hIgG1 (PDL-GEX Fuc−) and the fucose-reduced bispecific anti-PD-L1/TA-MUC1 hIgG1 (PM-PDL-GEX Fuc−) induce stronger CD69 expression on CD8 T cells compared to normal-fucosylated monospecific anti-PD-L1 hIgG1 (PDL-GEX H9D8) and bispecific anti-PD-L1/TA-MUC1 hIgG1 (PM-PDL-GEX H9D8. This is described in Example 11.

FIG. 12: FcyRs and its crucial role for the activation of T cells.

This allogeneic MLR with moDCs and isolated T cells shows that FcyR-binding plays a crucial role for the increased activation of T cells using a fucose-reduced anti-PD-L1 antibody. The increased T cell activation due to a fucose-reduced anti-PD-L1 hIgG1 (PDL-GEX Fuc−) was inhibited to a level comparable to the normal-fucosylated anti-PD-L1 hIgG1 (PDL-GEX H9D8) or non-glycosylated anti-PD-L1 hIgG1 with no/weak FcyR-binding capacity (Atezolizumab) due to addition of another fucose-reduced antibody with an irrelevant specificity (termed as block) (the antigen is not present in the MLR). This is described in Example 12.

FIG. 13: Measuring the maturation of dendritic cells.

In presence of a de-fucosylated anti-PD-L1 hIgG1 dendritic cells show a more mature phenotype compared to a normal-fucosylated anti-PD-L1 hIgG1. In presence of a fucose-reduced anti-PD-L1 hIgG1 (PDL-GEX Fuc−), moDCs show less expression of CD14 (A) compared to a normal-fucosylated anti-PD-L1 hIgG1 (PDL-GEX H9D8). In contrast, CD16 (FcγRIII) (B) and the co-stimulatory molecules CD40 (C) and CD86 (E), and the DC-marker CD83 (D) were expressed in higher levels in presence of a fucose-reduced anti-PD-L1 hIgG1 compared to a normal-fucosylated anti-PD-L1 hIgG1. This is described in Example 13.

FIG. 14: Activation of T cells measured by cytotoxicity.

Activation of T cells with PDL-GEX Fuc− resulted in increased cytotoxicity compared to PDL-GEX H9D8, Atezolizumab and medium control (medium control=T cells after a MLR without addition of test antibody). This effect was shown with T cells from two different healthy volunteers ((A)=donor 2, (B)=donor 3, which refer to the same donor as used in FIG. 9). This is described in Example 14.

FIG. 15: T cell activation using anti-PD-L1 hIgG1 with different amounts of core-fucosylation.

Activation of T cells with PDL-GEX was dependent on the amount of core-fucosylation as determined by the expression of CD137 (A) and CD25 (B) on CD8⁺ T cells. Medium and Atezolizumab (TECENTRIQ) served as controls. This is described in Example 15.

FIG. 16: Comparable antigen binding of anti-PD-L1 antibodies with mutations in their F_{c} part.

No obvious difference in PD-L1 binding was observed between PDL-GEX H9D8 (non-mutated), PDL-GEX H9D8 mut1 comprising three amino acid changes: S239D, 1332E and G236A according to EU nomenclature in the F_{c} part and PDL-GEX H9D8 mut2 comprising five amino acid changes: L235V, F243L, R292P, Y300L and P396L according to EU nomenclature. This is described in Example 16.

FIG. 17: Increased FcyRIIIa engagement of anti-PD-L1 antibodies with mutations in their F_{c} part.

PM-PDL-GEX H9D8 mut1 and PM-PDL-GEX H9D8 mut2 show increased binding to FcyRIIIa compared to the non-mutated PDL-GEX H9D8 visualized by the shift to lower effective concentrations. This is described in Example 17.

FIG. 18: Increased T cell activation of anti-PD-L1 antibodies with mutations in their Fc part,

PM-PDL-GEX mut1 and PDL-GEX mut2 show increased T cell activation in comparison to PDL-GEX H9D8 (non-mutated) demonstrating that enhanced T cell activation can be achieved by using either a de-fucosylated anti-PD-L1 antibody (PDL-GEX Fuc−) or by using anti-PD-L1 antibodies comprising sequence mutations leading to enhanced binding FcyRIIIa. This is described in Example 18.

FIG. 19: Enhanced T cell activation due to a de-fucoslyated anti-PD-L1 antibody visualized by proliferation.

The de-fucosylated anti-PD-L1 antibody (PDL-GEX Fuc−) shows increased proliferation of CD8 T cells compared to normal-fucosylated anti-PD-L1 antibody (PDL-GEX H9D8) and compared to a non-glycosylated anti-PD-L1 (Atezolizumab). This is described in Example 19.

FIG. 20: Enhanced T cell activation in presence of cancer cells.

A de-fucosylated anti-PD-L1 (PDL-GEX Fuc−) and de-fucosylated bispecific anti-PD-L1/TA-MUC1 antibody (PM-PDL-GEX Fuc−) were compared for their ability to induce T cell activation in presence of cancer cells in a MLR. However, the augmented activation by PDL-GEX Fuc− and PM-PDL-GEX Fuc− were observed in presence of all cancer cell lines tested. This is described in Example 20.

FIG. 21: PDL-GEX CDR mutants show comparable binding and blocking capacity compared to the non-mutated counterpart.

A) Fucose-reduced PDL-GEX having different mutations in the CDRs of the V_{H} domain binding to PD-L1 such as:

PDL-GEX Fuc− CDRmut a (SEQ ID NO. 60+SEQ ID NO. 68)

PDL-GEX Fuc− CDRmut b (SEQ ID NO. 62+SEQ ID NO. 69)

PDL-GEX Fuc− CDRmut c (SEQ ID NO. 63+SEQ ID NO. 70)

PDL-GEX Fuc− CDRmut d (SEQ ID NO. 64)

PDL-GEX Fuc− CDRmut e (SEQ ID NO. 65+SEQ ID NO. 71)

PDL-GEX Fuc− CDRmut f (SEQ ID NO. 66+SEQ ID NO. 72)

PDL-GEX Fuc− CDRmut g (SEQ ID NO. 63+SEQ ID NO. 72)

PDL-GEX Fuc− CDRmut h (SEQ ID NO. 67+SEQ ID NO. 74)

PDL-GEX Fuc− CDRmut i (SEQ ID NO. 63+SEQ ID NO. 68)

also show comparable PD-L1 binding capacity to the non-mutated PDL-GEX Fuc− using PD-L1 expressing Du-145 cells and flow cytometric analysis. B) The CDR mutants of the fucose-reduced PDL-GEX (see A) also show comparable blocking capacity to the non-mutated PDL-GEX Fuc− using PD-L1/PD1 blocking ELISA. This is described in Example 21.

FIG. 22: PM-PDL-GEX CDR mutants show comparable binding and blocking capacity compared to the non-mutated counterpart.

A) Fucose-reduced PM-PDL-GEX having different mutations in the CDRs of the V_{H} domain of the scFᵥ region binding to PD-L1, such as PM-PDL-GEX Fuc− CDRmut a (SEQ ID NO. 64), or PM-PDL-GEX Fuc− CDRmut b (SEQ ID NO. 66+SEQ ID NO. 72), show comparable PD-L1 binding capacity to the non-mutated PM-PDL-GEX Fuc− using PD-L1 antigen ELISA. B) The CDR mutants of the fucose-reduced PM-PDL-GEX also show comparable blocking capacity to the non-mutated PM-PDL-GEX Fuc− using PD-L1/PD1 blocking ELISA. C) Fucose-reduced PM-PDL-GEX having different mutations in the CDRs of the V_{H} domain show comparable TA-MUC1 binding capacity to the non-mutated PM-PDL-GEX Fuc− using TA-MUC1 expressing T-47D and flow cytometric analysis. This is described in Example 22.

FIG. 23: PM-PDL-GEX CDR mutants show comparable enhanced activation of CD8 T cells to the non-mutated counterparts.

Fucose-reduced PM-PDL-GEX having different mutations in the CDRs of the V_{H} domain of the scFᵥ region binding to PD-L1, such as PM-PDL-GEX Fuc− CDRmut a (SEQ ID No. 64), or PM-PDL-GEX Fuc− CDRmut b (SEQ ID NO. 66+SEQ ID NO. 72) show comparable enhanced CD8 T cell activation (CD25+ cells of CD8 T cells) to the non-mutated PM-PDL-GEX Fuc−. The CDR mutated PM-PDL-GEX H9D8 variants activated CD8 T cells comparable to non-mutated PM-PDL-GEX H9D8. This is described in Example 23.

### DETAILED DESCRIPTION OF THE INVENTION

The solution of the present invention is described in the following, exemplified in the appended examples, illustrated in the Figures and reflected in the claims.

The present invention provides a glycosylated antibody, which essentially lacks core-fucosylation and effects enhanced T cell activation in comparison to a reference antibody, which is glycosylated including more than 80% core-fucosylation.

The antibody of the present invention may be considered as a fucose-reduced monospecific anti-PD-L1 hIgG1 and a fucose-reduced bispecific anti-PD-L1/TA-MUC1 hIgG1, which are preferably obtainable from the cell line NM-H9D8-E6 (DSM ACC 2807), NM-H9D8-E6Q12 (DSM ACC 2856), or a cell or cell line derived therefrom. The monospecific and bispecific fucose-reduced antibody may comprise an F_{c} region and complex N-linked sugar chains bound to the F_{c} region, wherein among the total complex N-linked sugar chains bound to the F_{c} region, the content of 1,6-core-fucose for the fucose-reduced antibodies is from 0% to 80%.

Preferably, the host cell of the invention may be the cell, cells or cell line NM-H9D8-E6 (DSM ACC 2807) and/or NM-H9D8-E6Q12 (DSM ACC 2856), which grow and produce said fucose-reduced monospecific and fucose-reduced bispecific antibody of the invention under serum-free conditions. Also it may be preferred hereunder cells growing under serum-free conditions, wherein the nucleic acid encoding said fucose-reduced monospecific and fucose-reduced bispecific antibodies may be introduced in these cells and wherein said fucose-reduced monospecific and fucose-reduced bispecific antibodies may be isolated under serum-free conditions.

The monospecific, fucose-reduced antibody preferably refers to anti-PDL1-GEX Fuc− (short: PDL-GEX-Fuc−) and the bispecific, fucose-reduced antibody to the bispecific PankoMab-antiPDL1-GEX Fuc− (short: PM-PDL-GEX-Fuc−). This nomenclature can be used interchangeably.

The monospecific and bispecific fucose-reduced antibodies of the present invention were tested and compared to reference antibodies with regard to core-fucosylation, PD-L1 blocking capacity, binding to FcγRIIIa, binding to cells expressing TA-MUC1 and/or PD-L1, ADCC activity and T cell activation. As a reference antibody a normal-fucosylated monospecific anti-PDL-GEX (short: PDL-GEX-H9D8) and a normal-fucosylated bispecific anti-PM-PDL-GEX (short: PM-PDL-GEX H9D8) were used, which are glycosylated including more than 80% core-fucosylation and are preferably obtainable from CHOdhfr-(ATCC No. CRL-9096). Again, this nomenclature can be used interchangeably.

First, N-glycosylation of monospecific antibodies PDL-GEX H9D8 and PDL-GEX Fuc− and of bispecific antibodies PM-PDL-GEX H9D8 and PM-PDL-GEX Fuc− was analyzed by HILIC-UPLC-HiResQToF MSMS. The relative molar amounts of the core fucosylated N-glycans of monospecific antibodies PDL-GEX H9D8 and PDL-GEX Fuc− and of bispecific antibodies PM-PDL-GEX H9D8 and PM-PDL-GEX Fuc− are illustrated in FIG. 1.

The normal-glycosylated monospecific PDL-GEX H9D8 and the bispecific PM-PDL-GEX H9D8 may contain more than 80% core fucosylated N-glycans (core-fucosylation). The present invention envisages normal-glycosylated antibodies containing preferably more than 80% less than 100% core fucosylated N-glycans. The normal-glycosylated antibodies of the present invention may preferably contain about 81% to 100%, 85% to 95% fucosylated N-glycans or 90% to 95% fucosylated N-glycans. The normal-fucosylated antibodies of the present invention may contain more than 80%, 81%, 82%, 83%, 84%, 85%, 86%, 87%, 88%, 89%, 90%, 91%, 92%, 93%, 94%, 95%, 96%, 97%, 98%, 99%, or even 100% fucosylated N-glycans, preferably about 92% core fucosylated N-glycans for the PDL-GEX H9D8 antibody and preferably about 91% core fucosylated N-glycans for the PM-PDL-GEX H9D8. These antibodies having more than 80% core fucosylated N-glycans may therefore refer to normal-fucosylated antibodies.

The fucose-reduced monospecific PDL-GEX Fuc− and the bispecific PM-PDL-GEX Fuc− contain only low percentages of core fucosylated N-glycans. The present invention provides fucose-reduced antibodies preferably being from 0% to 80% fucosylated. The fucose-reduced antibodies of the present invention may preferably contain about 0% to 80%, 0% to 75%, 0% to 70%, 0% to 65%, 0% to 60%, 0% to 55%, 0% to 50%, 0% to 45%, 0% to 40%, 0% to 35%, 0% to 30%, 0% to 25%, 0% to 20%, 0% to 15%, 0% to 10% or 10% to 50%, 15% to 50%, 20% to 50%, 25% to 50%, 30% to 50%, 35% to 50%, 40% to 50%, 45% to 50% or 1% to 20%, 1% to 15%, 1% to 10%, 1% to 5% or 5% to 30%, 5% to 20%, 5% to 15% or 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10% fucosylated N-glycans. The fucose-reduced antibodies of the present invention may preferably contain 0%, 1%, 2%, 3%, 4%, 5%, 6%, 7%, 8%, 9%, 10%, 11%, 12%, 13%, 14%, 15%, 16%, 17%, 18%, 19%, 20.0%, 21%, 22%, 23%, 24%, 25%, 26%, 27%, 28%, 29%, 30%, 40%, 41%, 42%, 43%, 44%, 45.0%, 46%, 47%, 48%, 49%, 50%, 51%, 52%, 53%, 54%, 55%, 56%, 57%, 58%, 59%, 60%, 61.0%, 62%, 63%, 64%, 65%, 66%, 67%, 68%, 69%, 70%, 71%, 72%, 73%, 74%, 75%, 76%, 77%, 78%, 79%, or even 80% fucosylated N-glycans. More preferably, the fucose-reduced antibodies of the present invention may contain below 5% fucosylated N-glycans. Most preferably, about 4% fucosylated N-glycans for the PDL-GEX Fuc− antibody and about 1% fucosylated N-glycans for the PM-PDL-GEX Fuc− antibody. These antibodies being from 0% to 80% fucosylated may therefore refer to fucose-reduced antibodies. Additionally, the monospecific and bispecific fucose-reduced antibodies may have at least a 5% lower value of fucosylation compared to the same amount of antibody isolated from ATCC No. CRL-9096 (CHOdhfr-) when expressed therein.

Further, two different competitive ELISAs were applied in the present invention to analyze the potential of an anti-PD-L1 antibody and an antibody being capable of binding to TA-MUC1 and binding to PD-L1 with its scFᵥ region to inhibit the interaction of PD-L1 with its binding partners, PD-1 and CD80.

First, a fucose-reduced PDL-GEX Fuc− and a fucose-reduced bispecific PM-PDL-GEX Fuc− were compared to their normal-fucosylated counterparts PDL-GEX H9D8 and PM-PDL-GEX H9D8 in the PD-L1/PD-1 blocking ELISA. Concentration-dependent blocking of PD-1 binding was detected for all four variants tested. No difference between normal- and fucose-reduced monospecific anti-PD-L1 hIgG1, and normal- and fucose-reduced bispecific anti-PD-L1/TA-MUC1 hIgG1, respectively, was detected (FIG. 2A). Second, a related blocking ELISA was developed as described above, but instead of PD-1 CD80 ligand was used. All four variants tested showed effective inhibition of the interaction between PD-L1 and CD80 and no obvious difference between the glycosylation variants (fucose-reduced vs. normal-fucosylated) was detected (FIG. 2B). As a conclusion, the fucose-reduced antibodies show comparable blocking capacity compared to their normal-fucosylated counterparts.

These results were confirmed by the PD-1/PD-L1 blockade bioassay (Promega) which is a bioluminescent cell-based assay that can be used to measure the potency of antibodies designed to block the PD-1/PD-L1 interaction. A fucose-reduced and a normal-fucosylated bispecific anti-PD-L1/TA-MUC1 hIgG1 show comparable results in a cell based PD-1/PD-L1 blockade bioassay (FIG. 7).

Additionally, it was further shown that fucose-reduced PDL-GEX having different mutations in the CDRs of the V_{H} domain may also show comparable PD-L1 binding capacity to the non-mutated PDL-GEX Fuc−. The mutants of the fucose-reduced PDL-GEX may also show comparable blocking capacity to the non-mutated PDL-GEX Fuc− Preferably, comprising monospecific PD-L1 antibodies comprising mutations in the CDRs of the V_{H} domain, thus having the amino acid sequences as shown in SEQ ID NO. 60 (having a mutation of phenylalanine to isoleucine at position 29 according to Kabat-numbering in the CDR1 of the V_{H} domain) and 68 (having a mutation of serine to threonine at position 52 according to Kabat-numbering in the CDR2 of the V_{H} domain), or having the amino acid sequences as shown in SEQ ID NO. 62 (having a mutation of glycine to alanine at position 26 according to Kabat-numbering in the CDR1 of the V_{H} domain) and 69 (having a mutation of alanine to glycine at position 49 according to Kabat-numbering in the CDR2 of the V_{H} domain), or having the amino acid sequences as shown in SEQ ID NO. 63 (having a mutation of isoleucine to methionine at position 34 according to Kabat-numbering in the CDR1 of the V_{H} domain) and 70 (having a mutation of isoleucine to leucine at position 51 according to Kabat-numbering in the CDR2 of the V_{H} domain), or having the amino acid sequences as shown in SEQ ID NO. 64 (having a mutation of glycine to alanine at position 26 according to Kabat-numbering and having a mutation of aspartic acid to glutamic acid at position 31 according to Kabat-numbering in the CDR1 of the V_{H} domain), or having the amino acid sequences as shown in SEQ ID NO. 65 (having a mutation of aspartic acid to glutamic acid at position 31 according to Kabat-numbering in the CDR1 of the V_{H} domain) and 71 (having a mutation of valine to leucine at position 63 according to Kabat-numbering in the CDR2 of the V_{H} domain), or having the amino acid sequences as shown in SEQ ID NO. 66 (having a mutation of threonine to serine at position 28 according to Kabat-numbering in the CDR1 of the V_{H} domain) and 72 (having a mutation of serine to threonine at position 62 according to Kabat-numbering in the CDR2 of the V_{H} domain), or having the amino acid sequences as shown in SEQ ID NO. 63 (having a mutation of isoleucine to methionine at position 34 according to Kabat-numbering in the CDR1 of the V_{H} domain) and 72 (having a mutation of serine to threonine at position 62 according to Kabat-numbering in the CDR2 of the V_{H} domain), or having the amino acid sequences as shown in SEQ ID NO. 67 (having a mutation of serine to threonine at position 32 according to Kabat-numbering in the CDR1 of the V_{H} domain) and 74 (having a mutation of serine to threonine at position 56 according to Kabat-numbering in the CDR2 of the V_{H} domain), or having the amino acid sequences as shown in SEQ ID NO. 63 (having a mutation of isoleucine to methionine at position 34 according to Kabat-numbering in the CDR1 of the V_{H} domain) and 68 (having a mutation of serine to threonine at position 52 according to Kabat-numbering in the CDR2 of the V_{H} domain) (FIGS. 21A and B).

These data reveal that targeting cells expressing PD-L1 may be achieved with fucose-reduced and normal-fucosylated monospecific and bispecific antibodies of the present invention and/or with fucose-reduced monospecific antibodies having different CDR mutations in the V_{H} domain of said antibodies of the present invention.

Additionally, for further characterization of the fucose-reduced antibodies with regard to binding to TA-MUC1 expressed on tumor cells, the binding properties of normal-fucosylated and fucose-reduced bispecific PM-PDL-GEX H9D8 and Fuc− were analyzed by flow cytometry. The mamma carcinoma cell line ZR-75-1 with strong TA-MUC1 expression, but only minimal or absent PD-L1 expression was used to determine TA-MUC1 binding. Both, the fucose-reduced and the normal-fucosylated bispecific anti-PD-L1/TA-MUC1 hIgG1 showed comparable binding to TA-MUC1 (FIG. 3).

Additionally, it was further shown that fucose-reduced PM-PDL-GEX having different mutations in the CDRs of the V_{H} domain of the scFᵥ region binding to PD-L1, preferably having the amino acid sequence as shown in SEQ ID NO. 64 (having a mutation of glycine to alanine at position 26 according to Kabat-numbering and having a mutation of aspartic acid to glutamic acid at position 31 according to Kabat-numbering in the CDR1 of the V_{H} domain) or having the amino acid sequences as shown in SEQ ID NO. 66 (having a mutation of threonine to serine at position 28 according to Kabat-numbering in the CDR1 of the V_{H} domain) and 72 (having a mutation of serine to threonine at position 62 according to Kabat-numbering in the CDR2 of the V_{H} domain), may show comparable PD-L1 binding capacity, comparable blocking capacity of PD-L1/PD1 interaction and comparable TA-MUC1 binding capacity to the non-mutated PM-PDL-GEX (FIGS. 22A, B and C).

These data reveal that targeting tumor cells expressing TA-MUC1 may be achieved with fucose-reduced and normal-fucosylated bispecific antibodies of the present invention and/or with fucose-reduced bispecific antibodies having different CDR mutations in the V_{H} domain of the scFᵥ region binding to PD-L1 of said antibodies of the present invention preferably having the amino acid sequence as shown in SEQ ID NO. 64 or having the amino acid sequences as shown in SEQ ID NO. 66 and 72 as indicated above.

In addition to the findings above, it was found that the major difference between the fucose-reduced variants of a monospecific anti-PD-L1 hIgG1 and a bispecific anti-PD-L1/TA-MUC1 hIgG1 was the increased binding to FcyRIIIa compared to the normal-fucosylated variants. In order to characterize binding of the antibody F_{c} part to FcγRIIIa on a molecular level, a new assay using a bead-based technology of Perkin Elmer (AlphaScreen®) was developed. The fucose-reduced PDL-GEX Fuc− has a decreased EC50 value compared to the normal-fucosylated PDL-GEX H9D8 demonstrating ˜5-fold enhanced binding to FcγRIIIa of the fucose-reduced variant compared to the normal-fucosylated variant.

The bispecific fucose-reduced and normal-fucosylated anti-PD-L1/TA-MUC1 hIgG1 were not compared in the same experiment, but they were quantitatively compared by calculation of a relative potency compared to a normal-fucosylated reference antibody. The relative potency refers to the EC50 of the reference antibody divided by EC50 of the test antibody. For the bispecific normal-fucosylated PM-PDL-GEX H9D8 a relative potency of 1.9 was determined. In contrast, the relative potency of the bispecific fucose-reduced PM-PDL-GEX Fuc− was determined as 10.4. From that, the binding to FcγRIIIa is enhanced by ˜5-fold for the fucose-reduced variant compared to the normal-fucosylated counterpart (FIG. 4).

Further, another difference between the fucose-reduced and the normal-fucosylated antibodies was found. The fucose-reduced monospecific anti-PD-L1 hIgG1 and the fucose-reduced bispecific anti-PD-L1/TA-MUC1 hIgG1 show increased killing of TA-MUC+ and PD-L1+ tumor cells compared to their normal-fucosylated counterparts.

First of all, ADCC was analyzed against the breast cancer cell line ZR-75-1 which expresses high levels of TA-MUC1 and only marginal levels of PD-L1. As expected, due to increased binding to FcγRIIIa, the fucose-reduced bispecific PM-PDL-GEX Fuc− showed strongly enhanced ADCC activity compared to the normal-fucosylated bispecific anti-PD-L1/TA-MUC1 hIgG1 (FIG. 5A). This data implicates that ADCC may be enhanced against TA-MUC1⁺ cancer cells by applying the fucose-reduced bispecific PM-PDL-GEX Fuc− antibody.

Second, the prostate carcinoma cell line DU-145 strongly expressing PD-L1 and having moderate TA-MUC1 expression was used for further investigation of killing of also PD-L1+ tumor cells. It was found again, that the fucose-reduced monospecific PDL-GEX Fuc− and the fucose-reduced bispecific PM-PDL-GEX Fuc− mediated strongly enhanced ADCC against PD-L1 positive tumor cells compared to their normal-fucosylated counterparts (FIG. 5D). This data implicate that ADCC may be enhanced against PD-L1⁺ cancer cells by applying the fucose-reduced monospecific PDL-GEX Fuc− and the bispecific PM-PDL-GEX Fuc− antibody.

PD-L1 is reported to be expressed not exclusively on tumor cells but also on different immune cells, e.g. monocytes or B cells. Since fucose-reduced monospecific anti-PD-L1 and fucose-reduced bispecific anti-PD-L1/TA-MUC1 show strongly increased ADCC effects against tumor cells compared to their normal-fucosylated counterparts, it could be expected that they also mediate ADCC against PD-L1+ immune cells. Since monocytes and B cells are described to express PD-L1, both immune cell populations were analyzed in a FACS based ADCC assays as potential target cells.

Surprisingly, no ADCC effect mediated by fucose-reduced monospecific anti-PD-L1 and fucose-reduced bispecific anti-PD-L1/TA-MUC1 against immune cells such as B cells and monocytes was detected (FIGS. 6A and B).

Further, the experiments described in Example 8 show that a fucose-reduced and a normal-fucosylated bispecific anti-PD-L1/TA-MUC1 hIgG1 and a fucose-reduced anti-PD-L1 hIgG1 induce comparable IL-2 in an allogeneic mixed lymphocyte reaction (MLR) (FIG. 8B).

The mixed lymphocyte reaction (MLR) is a functional assay which was established to analyze the effect of PD-L1 blocking antibodies on the suppression of PD-1 expressing T cells by PD-L1 expressing antigen presenting cells. The assay measures the response of T cells from one donor as responders to monocyte-derived dendritic cells (moDCs) from another donor as stimulators (=allogenic MLR).

The present inventors also surprisingly found that a fucose-reduced monospecific anti-PD-L1 hIgG1 and fucose-reduced bispecific anti-PD-L1/TA-MUC1 hIgG1 may show enhanced T cell activation measured in an allogeneic mixed lymphocyte reaction (MLR) in comparison to the normal-fucosylated counterparts and an anti-PD-L1 antibody called “Atezolizumab” as another reference antibody (FIGS. 9A, B and C). Thus, also comprised by the present invention is an antibody, which effects enhanced T cell activation measured in an allogeneic mixed lymphocyte reaction (MLR) in comparison to a reference antibody being glycosylated including more than 80% core-fucosylation.

CD8 T cells (CD3⁺CD8⁺ cells) of allogeneic MLRs with moDCs and isolated T cells in presence of test antibody (1 μg/ml test antibody) were analyzed for activation via expression of CD25 by flow cytometry. Results obtained with T cells from different donors demonstrate that a fucose-reduced PDL-GEX Fuc− and a fucose-reduced bispecific PM-PDL-GEX Fuc− may induce enhanced T cell activation compared to normal-fucosylated monospecific PDL-GEX H9D8 and bispecific PM-PDL-GEX H9D8, also compared to another anti-PD-L1 antibody such as Atezolizumab. This latter reference antibody called “Atezolizumab” may have no or weak FcyR-binding capacity and is non-glycosylated. An increased T cell activation due to a fucose-reduced anti-PD-L1 in comparison to a normal-fucosylated anti-PD-L1 was also confirmed in FIG. 14. In order to analyze whether increased T cell activation due to a fucose-reduced anti-PD-L1 results in a benefit in functionality, T cells which were activated in a allogeneic MLR in absence or presence of PDL-GEX H9D8, PDL-GEX Fuc− and Atezolizumab were harvested and afterwards their cytotoxic capacity was determined using a europium release assay.

The fact that fucose-reduced anti-PD-L1 and anti-PD-L1/TA-MUC1 antibodies may induce increased T cell activation is surprising, since no differences between the glycosylation variants were seen in the blocking ELISA (see Example 2), in the PD-1/PD-L1 blockade bioassay (see Example 7) and in the IL-2 secretion (see Example 8). Increased activation of T cells due to fucose-reduced monospecific anti-PD-L1 hIgG1 and fucose-reduced bispecific anti-PD-L1/TA-MUC1 hIgG1 is observed with T cells of different donors and is again expected to be a surprising effect.

This finding that fucose-reduced monospecific anti-PD-L1 and bispecific anti-PD-L1/TA-MUC1 hIgG1 may induce enhanced CD8 T cell activation is important, since CD8 T cells represent cytotoxic T cells which play a crucial role in the anti-tumor response and have the capacity to directly kill cancer cells. After the treatment with a fucose-reduced monospecific PD-L1 antibody and a fucose-reduced bispecific antibody being capable of binding PD-L1 and TA-MUC1, increased T cell activation may occur during cancer diseases, inflammatory diseases, virus infectious diseases and autoimmune diseases.

It was further shown that enhanced T cell activation due to a de-fucoslyated anti-PD-L1 antibody and a de-fucosylated bispecific anti-PD-L1/TA-MUC1 antibody may also be observed in presence of cancer cells, such as HSC-4, ZR-75-1, Ramos cancer cells in a MLR (FIG. 20).

The present invention may provide a monospecific PD-L1 antibody (e.g. PDL-GEX Fuc−) effecting enhanced T cell activation in comparison to (i) a reference PD-L1 antibody being glycosylated including more than 80% core-fucosylation (e.g. PDL-GEX-H9D8) and in comparison to (ii) a reference antibody being non-glycosylated (e.g. Atezolizumab). Additionally, the present invention may provide a bispecific antibody (e.g. PM-PDL-GEX Fuc−) being capable of binding to TA-MUC1 and PD-L1 with its scFᵥ regions and effecting enhanced T cell activation in comparison to (i) a reference antibody being capable of binding to TA-MUC1 and PD-L1 and being glycosylated including more than 80% core-fucosylation (e.g. PM-PDL-GEX-H9D8).

In another allogeneic MLR isolated T cells or PBMCs were cultivated with moDCs in presence of a test antibody. Flow cytometric analysis shows that the PDL-GEX Fuc− and the PM-PDL-GEX Fuc− induced stronger CD8⁺ T cell activation compared to normal-fucosylated monospecific anti-PD-L1 hIgG1 or to a bispecific anti-PD-L1/TA-MUC1 hIgG1 and compared to an anti-PD-L1 hIgG1 such as Atezolizumab measured by expression of CD25 and CD137 on CD3⁺CD8⁺ cells using either T cells (FIGS. 10A and B) or Peripheral Blood Mononuclear Cells (PBMCs) (FIGS. 10C and D) as responder cells in the MLR. Cultivation of moDCs with PBMCs additionally leads to increased CD4 T cell activation (CD3⁺CD8⁻ cells ergo CD4 T cells) due to the fucose-reduced monospecific PDL-GEX Fuc− and the fucose-reduced bispecific PM-PDL-GEX Fuc− measured by expression of CD25 (FIG. 10E) and CD137 (FIG. 10F), which was not observed earlier in MLRs using isolated T cells. Interestingly, the usage of PBMCs, which contain NK cells, instead of isolated T cells shows that NK cells or a potential NK cell-mediated ADCC effect on PD-L1+ cells has no negative impact on T cell activation.

To complete the findings above, enhanced T cell activation due to the de-fucosylated anti-PD-L1 antibody (PDL-GEX Fuc−) may also be visualized by proliferation. The PDL-GEX Fuc− antibody may show increased proliferation of CD8 T cells compared to the normal-fucosylated anti-PD-L1 antibody (PDL-GEX H9D8) and compared to an anti-PD-L1 being non-glycosylated (Atezolizumab) (FIG. 19).

Further, these data were confirmed and even extended by the finding in another allogenic MLR that a fucose-reduced anti-PD-L1 hIgG1 (PDL-GEX Fuc−) and fucose-reduced bispecific anti-PD-L1/TA-MUC1 hIgG1 (PM-PDL-GEX Fuc−) may also increase CD69 expression on T cells compared to their normally fucosylated couterparts (FIG. 11). Besides CD25 and CD137, CD69 is an additional activation marker which is stronger induced after treatment with monospecific and/or bispecific fucose-reduced antibodies.

Further, the present invention discloses that T cell activation may be detectable by the expression level of CD25, CD69 and/or CD137. Having activated T cells detectably by the expression level of CD137 and/or CD25, in this context or elsewhere herein, means that at least 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55% or 60%, or from 8% to 60%, 8% to 55%, 8% to 50%, 8% to 45%, 8% to 40%, 8% to 35%, 8% to 30%, 8% to 25%, 8% to 24%, 8% to 23%, 8% to 22%, 8% to 21%, 8% to 20%, 8% to 19%, 8% to 18%, 8% to 17%, 8% to 16%, 8% to 15% CD137⁺ and/or CD25⁺ T cells of all measured CD8⁺ T cells are detected. Preferably, having activated T cells detectably by the expression level of CD25, in this context, means that 8% to 25%, 8% to 24%, 8% to 23%, 8% to 22%, 8% to 21%, or 8% to 20% CD25⁺ T cells of all measured CD8⁺ T cells are detected. Preferably, having activated T cells detectably by the expression level of CD137, in this context, means that 8% to 20%, 8% to 19%, 8% to 18%, 8% to 17%, 8% to 16%, 8% to 15% CD137⁺ T cells of all measured CD8⁺ T cells are detected. Said activation of at least 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55% or 60%, or from 8% to 60%, 8% to 55%, 8% to 50%, 8% to 45%, 8% to 40%, 8% to 35%, 8% to 30%, 8% to 25%, 8% to 24%, 8% to 23%, 8% to 22%, 8% to 21%, 8% to 20%, 8% to 19%, 8% to 18%, 8% to 17%, 8% to 16%, 8% to 15% CD137⁺ and/or CD25⁺ T cells of all CD8⁺ T cells is achieved by using antibodies of the present invention, which are from 0% to 80%, 0% to 75%, 0% to 70%, 0% to 65%, 0% to 60%, 0% to 55%, 0% to 50%, 0% to 45%, 0% to 40%, 0% to 35%, 0% to 30%, 0% to 25%, 0% to 20%, 0% to 15%, 0% to 10%, 0% to 5% fucosylated, preferably from 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10% fucosylated or below 5% fucosylated, most preferably 4% fucosylated (FIG. 15). Said activation of at least 15%, 16%, 17%, 18%, 19%, 20%, 21%, 22%, 23%, 24%, 25%, 30%, 35%, 40%, 45%, 50%, 55% or 60% CD137⁺ and/or CD25⁺ T cells of all CD8⁺ T cells is achieved by using antibodies of the present invention, which are from 0% to 80%, 0% to 75%, 0% to 70%, 0% to 65%, 0% to 60%, 0% to 55%, 0% to 50%, 0% to 45%, 0% to 40%, 0% to 35%, 0% to 30%, 0% to 25%, 0% to 20%, 0% to 15%, 0% to 10%, 0% to 5% fucosylated, preferably from 4% to 80%, 4% to 75%, 4% to 70%, 4% to 65%, 4% to 60%, 4% to 55%, 4% to 50%, 4% to 45%, 4% to 40%, 4% to 35%, 4% to 30%, 4% to 25%, 4% to 20%, 4% to 15%, 4% to 10% fucosylated or below 5% fucosylated, most preferably 4% fucosylated and have mutations in the CDRs of the V_{H} domain (of the scFᵥ region) binding to PD-L1 as indicated elsewhere herein. In general, 100.000 T cells are used, e.g. for a mixing trial as described in Example 15. Normally, T cells comprise CD4⁺ T cells (CD4) as well as CD8⁺ T cells (CD8) and a small amount of natural killer T cells (NKT). The amount of CD8⁺ T cells used may be achieved by applying literature references from the prior art regarding an amount of CD8⁺ T cells (CD45⁺CD3⁺CD8+) within total T cells (CD45⁺CD3+), which is preferably 36%. Using the preferred percentage amount of 36%, for example at least 8% CD137⁺ and/or CD25⁺ T cells of all measured CD8⁺ T cells means having for example at least 2880 CD137⁺ and/or CD25⁺ T cells (Valiathan et al., 2014, Immunobiology 219, 487-496). Same applies mutatis mutandis to other percent values as listed above.

To investigate how the specific and enhanced T cell activation may be induced, another allogeneic MLR with moDCs and isolated T cells was performed showing that FcyRs may play a crucial role for the increased activation of T cells using a fucose-reduced anti-PD-L1 antibody. Thus, the increased T cell activation may be considered as being connected with FcyR-binding capacity, preferably with FcyRIIIa-binding capacity, thus being indirectly linked to F_{c}-N-glycosylation.

The increased T cell activation due to a fucose-reduced anti-PD-L1 hIgG1 (PDL-GEX Fuc−) was inhibited to a level comparable to the normal-fucosylated anti-PD-L1 hIgG1 (PDL-GEX H9D8) or to the non-glycosylated anti-PD-L1 hIgG1 (Atezolizumab) due to addition of another fucose-reduced antibody with an irrelevant specificity (termed as block) (FIG. 12). This experiment described in Example 12 may demonstrate the important role of FcγRs in general for the increased T cell activation due to application of fucose-reduced anti-PD-L1 antibodies. Since it is known from Example 4 that fucose-reduced variants of monospecific anti-PD-L1 and bispecific anti-PD-L1/TA-MUC1 may show increased binding to FcyRIIIa compared to their normal-fucosylated counterparts, it is all the more persuasive that the specific receptor FcyRIIIa may be responsible for enhanced T cell activation. Consequently, T cell activation may be mediated through enhanced binding to FcyRI (CD64), FcyRII (CD32), including isoforms FcyRIIa, FcyRIIb, FcyRIIIc or FcyRIII (CD16), including isoforms FcyRIIIa or FcyRIIIb, preferably through enhanced binding to FcyRIIIa.

Finally, the fucose-reduced bispecific antibodies having different CDR mutations in the V_{H} domain of the scFᵥ region binding to PD-L1, preferably having the amino acid sequence as shown in SEQ ID NO. 64 (having a mutation of glycine to alanine at position 26 according to Kabat-numbering and having a mutation of aspartic acid to glutamic acid at position 31 according to Kabat-numbering in the CDR1 of the V_{H} domain) or having the amino acid sequences as shown in SEQ ID NO. 66 (having a mutation of threonine to serine at position 28 according to Kabat-numbering in the CDR1 of the V_{H} domain) and 72 (having a mutation of serine to threonine at position 62 according to Kabat-numbering in the CDR2 of the V_{H} domain) as indicated elsewhere herein, may further show comparable enhanced CD25 T cell activation to the non-mutated PM-PDL-GEX Fuc− (FIG. 23). These data reveal that fucose-reduced bispecific antibodies of the present invention and/or fucose-reduced bispecific antibodies having different CDR mutations in the V_{H} domain of the scFᵥ region binding to PD-L1, preferably having the amino acid sequence as shown in SEQ ID NO. 64 or having the amino acid sequences as shown in SEQ ID NO. 66 and 72 may also enhance T cell activation in comparison to a reference antibody being glycosylated including more than 80% core-fucosylation.

The present invention certainly enriches the prior art by providing an antibody of the present invention since activating T cells with a glyco-optimized antibody is a very encouraging approach for all kinds of diseases, which can be associated with T cell activation.

As an alternative approach to increase the FcyR-mediated effector function via glycosylation of the F_{c} region, as already discussed, efforts have focused on increasing the affinity of the F_{c} region via F_{c} engineering.

In general, antibody drug development focuses on engineering the top part of an antibody which is being responsible for binding to an antigen target. However, researchers at different locations such as Genentech, Xencor or Medlmmune take the approach by focusing on engineering the F_{c} region of an antibody, which is responsible for the natural immune functions of said antibody. Certain mutations within the F_{c} region, a selection of the amino acids that have been targeted for enhancing F_{c} effector functions, were identified being either directly or indirectly linked to an enhanced binding of Fc receptors, thus also an enhancement of cellular cytotoxicity (f.e. ADCC and/or ADCP). Researchers at Genentech identified the mutations S239D/A330L/1332E (Lazar et al., 2006, “Engineered antibody Fc variants with enhanced effector function”, PNAS 103, 4005-4010 and Shields et al., 2001, “High Resolution Mapping of the Binding Site on Human IgG1 for FcγRI, FcγRII, FcγRIII, and FcRn and Design of IgG1 Variants with Improved Binding to the FcγR”, J. Biol. Chem. 276, 6591-6604), Medlmmune identified the mutation F243L (Stewart et al., 2011, “A variant human IgG1-Fc mediates improved ADCC”, Protein Engineering, Design and Selection 24, 671-678) and Xencor identified G236A (Richards et al, 2008, “Optimization of antibody binding to FcγRIIa enhances macrophage phagocytosis of tumor cells”, Mol Cancer Ther 7, 2517-2527).

According to Lazar et al. (2006) different variants were constructed including single mutants S239D and 1332E, the double mutant S239D/1332E and the triple mutant S239D/1332E/A330L, expressed, purified and screened for FcyR affinity. Those variants, in particularly a combination of A330L with S239D/1332E, illustrate significant enhancement in binding to the specific FcyRIIIa receptor. Variants including double (S239D/1332E) mutants also provide significant increase in binding to the specific FcyRIIIa receptor. The S239D/1332E and S239D/1332E/A330L variants also provide substantial ADCC enhancements.

The present invention may comprise an antibody comprising one or more sequence mutations, wherein the binding of said antibody to FcyRIIIa may be increased compared to a non-mutated antibody. Those sequence mutations may be selected from S238D, S239D, 1332E, A330L, S298A, E333A, L334A, G236A, L235V, F243L, R292P, Y300L, V3051, and P396L, according to EU-nomenclature, wherein the numbering is according to the EU index as in Kabat. An antibody of the present invention comprising one or more sequence mutations from the ones listed above may be a monospecific PD-L1 antibody or a bispecific antibody being capable of binding to TA-MUC1 and binding to PD-L1 with its scFᵥ regions. Further, the present invention may also envisage a bispecific antibody being capable of binding to PD-L1 and binding to TA-MUC1 with its scFᵥ regions and comprising one or more sequence mutations from the ones listed above The antibody of the present invention not being de-fucosylated, but comprising one or more sequence mutations may enhance T cell activation in comparison to a reference antibody with no mutations. Single mutations selected from the sequence mutations listed above or double, triple, quadruple, quintuple mutations chosen from any sequence mutation listed above may lead to an increased binding to FcyRs, preferably to FcγRIIIa and thus to an enhanced T cell activation. In a specific embodiment, an antibody of the present invention comprising the triple mutation G236A/S239D/1332E in their F_{c} part or the quintuple mutation L235V/F243L/R292P/Y300L/P396L in their F_{c} part may be preferred. An antibody of the present invention comprising the triple mutation G236A/S239D/1332E or the quintuple mutation L235V/F243L/R292P/Y300L/P396L may be a normal-fucosylated monospecific PD-L1 antibody or a normal-fucosylated bispecific antibody being capable of binding to TA-MUC1 and binding to PD-L1 with its scFᵥ regions, which may exhibit an increased FcγRIIIa-binding and thus enhanced T cell activation. The present invention may further comprise a bispecific antibody being capable of binding to PD-L1 and binding to TA-MUC1 with its scFᵥ regions and comprising the triple mutation G236A/S239D/1332E and the quintuple mutation L235V/F243L/R292P/Y300L/P396L, which may exhibit an increased FcγRIIIa-binding and thus enhanced T cell activation.

It was clearly shown that even though two normal-fucosylated anti-PD-L1 antibodies, the first comprising three amino acid changes S239D, 1332E and G236A in the F_{c} part of the antibody (PDL-GEX H9D8 mut1) according to Kabat-numbering and the second comprising five amino acid changes: L235V, F243L, R292P, Y300L and P396L in the F_{c} part of the antibody according to Kabat-numbering (PDL-GEX H9D8 mut2) showed comparable antigen binding to their non-mutated counterpart (PDL-GEX H9D8) (FIG. 16), the antibodies showed increased FcyRIIIa engagement (FIG. 17) and increased T cell activation (FIG. 18). Thus, said activation of at least 15%, 16%, 17%, 18%, 19%, 20%, 21%, 22%, 23%, 24%, 25%, 30%, 35%, 40%, 45%, 50%, 55% or 60% CD137⁺ and/or CD25⁺ T cells of all CD8⁺ T cells is achieved by using antibodies of the present invention, which comprise the triple mutation G236A/S239D/1332E in their F_{c} part or the quintuple mutation L235V/F243L/R292P/Y300L/P396L in their F_{c} part.

The present invention may further comprise an antibody lacking F_{c} glycosylation, thus being non-glycosylated, and comprising one or more of said sequence mutations or any double, triple, quadruple, quintuple mutation chosen from any sequence mutation listed above, which may lead to increased binding to FcγRIIIa and thus to an enhanced T cell activation.

To sum it up, it is now known from the present invention that said PD-L1 antibody (PDL-GEX Fuc−) may be capable of enhancing T cell activation through enhanced binding to FcyR, preferably to FcyRIIIa of immune cells in comparison to (i) a PD-L1 antibody with no or weak FcyRIIIa-binding (f.e. Atezolizumab) and to (ii) a PD-L1 antibody with normal FcyRIIIa-binding (PDL-GEX-H9D8). It is also known from the present invention that said antibody being capable of binding to TA-MUC1 and binding to PD-L1 with its scFᵥ regions (PM-PDL-GEX Fuc−) may be capable of enhancing T cell activation through enhanced binding to FcyR, preferably to FcyRIIIa of immune cells in comparison to an antibody being capable of binding to TA-MUC1 and binding to PD-L1 with its scFᵥ regions (PM-PDL-GEX-H9D8) and having normal FcyRIIIa-binding. Same applies mutatis mutandis to FcyRI and/or FcyRII.

In other words said glycosylated, essentially de-fucosylated PD-L1 antibody may be capable of enhancing T cell activation through enhanced binding to FcyR, preferably to FcyRIIIa of immune cells in comparison to (i) a non-glycosylated PD-L1 antibody (f.e. Atezolizumab) and to (ii) a glycosylated, normal-fucosylated PD-L1 antibody (PDL-GEX-H9D8). The present invention may further contemplate a glycosylated, essentially de-fucosylated antibody being capable of binding to TA-MUC1 and binding to PD-L1 with its scFᵥ regions (PM-PDL-GEX-H9D8), which may be capable of enhancing T cell activation through enhanced binding to FcyR, preferably to FcyRIIIa of immune cells in comparison to a glycosylated, normal-fucosylated antibody being capable of binding to TA-MUC1 and binding to PD-L1 with its scFᵥ regions (PM-PDL-GEX-H9D8).

Additionally, the inventors found that in presence of a de-fucosylated anti-PD-L1 hIgG1 dendritic cells show a more mature phenotype compared to a normal-fucosylated anti-PD-L1 hIgG1 antibody. This was demonstrated by the expression of different markers using flow cytometry. CD16 (FcγRIII) and the co-stimulatory molecules CD40 and CD86, and the DC-marker CD83 were expressed in higher levels in presence of a de-fucosylated anti-PD-L1 hIgG1 compared to a normal-fucosylated anti-PD-L1 hIgG1 (FIGS. 13B, C, D and E) This experiment described in Example 13 shows that fucose-reduced anti-PD-L1 hIgG1 may have a positive effect on the maturation status of DCs, which may activate T cells in return, helping to determine T cell activation. Therefore, T cell activation may be considered as being accompanied by maturation of dendritic cells and/or expression of co-stimulatory molecules (e.g. CD40, CD86 etc.) and maturation markers such as CD83.

An enhanced T cell response via FcγRIIIa-dependent maturation of DCs may be determined by an antibody of the present invention characterized by the enhanced binding of the F_{c} region to FcγRs, preferably to FcγRIIIa on DCs.

To this end and in view of enhancing T cell activation with a PD-L1 antibody and/or an antibody being capable of binding to TA-MUC1 and binding to PD-L1 with its scFᵥ regions, the present invention may further encompass a PD-L1 antibody as described herein and/or an antibody being capable of binding to TA-MUC1 and binding to PD-L1 with its scFᵥ regions as described herein for use in therapy. In particular, the present invention may further encompass a PD-L1 antibody as described herein and/or an antibody being capable of binding to TA-MUC1 and binding to PD-L1 with its scFᵥ regions as described herein for use in a method for activating T cells. The activation of T cells may be for the treatment of cancer disease, inflammatory disease, virus infectious disease and autoimmune disease. Preferably, T cell activation is useful for the treatment of cancer disease.

Cancer disease may be selected from Thymic Carcinoma, Lymphoma incl. Hodgkin's Lymphoma, Malignant Solitary Fibrous Tumor of the Pleura (MSFT), Penile Cancer, Anal Carcinoma, Thyroid Carcinoma, Head and Neck Squamous Carcinoma (HNSC), Non-small cell lung cancer (NSCLC), Small Cell Lung Cancer (SCLC), Vulvar Cancer (squamous cell carcinoma), Bladder Cancer, Cervical Cancer, Non-Melanoma Skin Cancer, (Retro-) Peritoneal Carcinoma, Melanoma, Gastrointestinal Stromal Tumor (GIST), Malignant Pleural Mesothelioma, Renal Cell Carcinoma (RCC), Kidney Cancer, Hepatocellular Carcinoma (HCC), Esophageal and Esophagogastric Junction Carcinoma, Extrahepatic Bile Duct Adenocarcinoma, Male Genital Tract Malignancy, Small Intestinal Malignancy, Sarcoma, Pancreatic Adenocarcinoma, Stomach Cancer (Gastric Adenocarcinoma), Breast Carcinoma, Colorectal Cancer (CRC), Malignant Mesothelioma, Merkel Cell Carcinoma, Squamous Cell Cancers, Advanced Carcinoma, Prostate Cancer, Ovarian Cancer, Endometrial Cancer, Urothelial Carcinoma (UCC), Lung Cancer. Preferably, cancer disease may be selected from Melanoma, Carcinoma, Lymphoma, Sarcoma, and Mesothelioma including Lung Cancer, Kidney Cancer, Bladder Cancer, Gastrointestinal Cancer, Skin Cancer, Breast Cancer, Ovarian Cancer, Cervical Cancer, and Prostate Cancer, most preferably cancer disease may be Breast Cancer.

Further, the present invention may envisage the use of an antibody of the present invention, preferably a PD-L1 antibody and/or an antibody being capable of binding to TA-MUC1 and binding to PD-L1 with its scFᵥ regions, for the manufacture of a medicament for therapeutic application in cancer disease, inflammatory disease, virus infectious disease and autoimmune disease. Further, the present invention may encompass the use of an antibody of the present invention, preferably a PD-L1 antibody and/or an antibody being capable of binding to TA-MUC1 and binding to PD-L1 with its scFᵥ regions, for the manufacture of a medicament for activating T cells.

Additionally, the present invention may include a method of activating T cells in a subject comprising administering an effective amount of said antibody, preferably a PD-L1 antibody and/or an antibody being capable of binding to TA-MUC1 and binding to PD-L1 with its scFᵥ regions, to a subject in need thereof.

The present invention may further contemplate an antibody of the present invention for use in a method for activating T cells in a subject. An antibody of the present invention may be administered to a subject suffering from cancer disease and/or inflammatory disease and/or virus infectious disease and/or autoimmune disease. The subject may be any subject as defined herein, preferably a human subject. The subject is preferably in need of the administration of an antibody of the present invention. Preferably, the subject may be an animal, including birds. The animal may be a mammal, including rats, rabbits, pigs, mice, cats, dogs, sheep, goats, and humans. Most preferably, the subject is a human. In one embodiment, the subject is an adult.

### Definitions

The term “glycosylation” refers to two N-linked oligosaccharides at each conserved asparagine 297 (Asn297/N297), according to EU-nomenclature, in the CH₂ domains of the F_{c} region of an antibody. Here, glycosylation of a monospecific PD-L1 antibody and a bispecific antibody being capable of binding to TA-MUC1 and binding to PD-L1 with its scFᵥ regions, which are glycosylated, essentially lacking core-fucosylation (e.g. fucose-reduced antibodies such as PDL-GEX-Fuc− and PM-PDL-GEX Fuc−) as well as glycosylation of a normal-glycosylated antibody including more than 80% core-fucosylation (e.g. normal-fucosylated antibodies such as PDL-GEX-H9D8 and PM-PDL-GEX H9D8) preferably refer to human glycosylation.

The term “human glycosylation” refers to a known F_{c}-N-glycosylation having two N-linked oligosaccharides at each N297 in the CH₂ domains of the F_{c} region. The general structure of N-linked oligosaccharides, which glycosylated antibodies of the present invention contain may be complex-type and is described as follows: A mannosyl-chitobiose core (Man3GlcNAc2-Asn) with variations in the presence/absence of bisecting N-acetylglucosamine and the innermost core L-fucose (Fuc), which may be α-1.6-linked to the N-acetylglucosamine. Furthermore, the complex type N-glycosylation may be characterized by antennary N-acetylglucosamine linked to the mannosyl-chitobiose core (Man3GlcNAc2-Asn) with optional extension of the antenna by galactose and sialic acid moieties. The innermost core L-fucose of the present invention may be α-1.6-linked to the N-acetylglucosamine (GlcNac) of the N-linked oligosaccharide structure.

The term “N-linked oligosaccharides” refers to N-linked sugar chains/N-glycans bound to the F_{c} region, more specific it refers to N-linked sugar chains/N-glycans, which are bound to both CH₂ domains of the F_{c} region, preferably attached onto each N297 in both CH₂ domains of the F_{c} region. In total, the present invention comprises two N-linked oligosaccharides.

The term “normal-glycosylated antibody” refers to an antibody containing two N-linked oligosaccharides at each N297 in the CH₂ domains of the F_{c} region, thus being glycosylated. Further, normal-glycosylated antibodies of the present invention may comprise more than 80% α-1,6-core fucosylation as well. Therefore, normal-glycosylated antibodies of the present invention may refer to glycosylated antibodies, being normal-fucosylated. Here, normal-glycosylated antibodies may refer to a bifunctional monospecific PDL-GEX-H9D8 as well as to a trifunctional bispecific PM-PDL-GEX H9D8, which may be used as said reference antibodies. In this context, normal-glycosylated antibodies of the present invention may be obtainable from CHOdhfr− (ATCC No. CRL-9096).

The term “non-glycosylated antibody” may refer to an anti-PD-L1 antibody, no matter if such antibody is monospecific or bispecific, which may have no or weak FcyR-binding capacity, preferably FcyRIIIa-binding capacity, thus having reduced T cell activation. A non-glycosylated antibody does not contain two N-linked oligosaccharides at each N297 in the CH₂ domains of the F_{c} region, thus being non-glycosylated. Preferably, the Roche antibody “Atezolizumab” may be used as said reference antibody, which is non-glycosylated. This antibody is known to the skilled man in the art. Commonly, non-glycosylation in Atezolizumab is due to modification in the amino acid sequence of asparagine to alanine (aa297), according to EU-nomenclature.

The term “non-glycosylated” may also be used interchangeably with the term “aglycosylated” or nouns such as “aglycosylation” thereof.

The term “normal-fucosylated antibody” may refer to an antibody, no matter if such antibody is monospecific or bispecific, which may have a normal FcyR-binding capacity, preferably FcyRIIIa-binding capacity, thus having normal T cell activation. The normal-fucosylated antibodies of the present invention are glycosylated, having two N-linked sugar chains bound to the F_{c} region, wherein among the total complex N-linked sugar chains bound to the F_{c} region, the content of 1,6-core-fucose may be more than 80%. The normal-fucosylated antibodies of the present invention may contain more than 80% less than 100% core fucosylated N-glycans. The normal-glycosylated antibodies of the present invention may preferably contain about 81% to 100%, 85% to 95% fucosylated N-glycans or 90% to 95% fucosylated N-glycans. The normal-fucosylated antibodies of the present invention may contain more than 80%, 81%, 82%, 83%, 84%, 85%, 86%, 87%, 88%, 89%, 90%, 91%, 92%, 93%, 94%, 95%, 96%, 97%, 98%, 99%, or even 100% fucosylated N-glycans. Preferably, the term “normal-fucosylated antibody” may refer to the term “antibody being glycosylated including more than 80% core-fucosylation” or may refer to the term “glycosylated, normal-fucosylated antibody”. Here, a normal-fucosylated antibody may refer to a bifunctional monospecific PDL-GEX-H9D8 as well as a trifunctional bispecific PM-PDL-GEX H9D8 antibody

The term “fucose-reduced antibody” may refer to an antibody, no matter if such antibody is monospecific or bispecific, which may have an increased FcyR-binding capacity, preferably FcyRIIIa-binding capacity, thus having enhanced T cell activation. Fucose-reduced antibodies of the present invention contain two N-linked oligosaccharides at each N297 in the CH₂ domains of the F_{c} region, thus being glycosylated. Further, fucose-reduced antibodies of the present invention may comprise from 0% to 80% α-1,6-core fucosylation. In particular, fucose-reduced antibodies of the present invention comprise an F_{c} region and have two complex N-linked sugar chains bound to the F_{c} region, wherein among the total complex N-linked sugar chains bound to the F_{c} region, the content of 1,6-core-fucose may be from 0% to 80%. The fucose-reduced antibodies of the present invention may preferably contain about 0% to 70%, 0% to 60%, 0% to 50%, 0% to 40%, 0% to 30%, 0% to 20%, 0% to 10% or 10% to 50%, 15% to 50%, 20% to 50%, 25% to 50%, 30% to 50%, 35% to 50%, 40% to 50%, 45% to 50% or 1% to 20%, 1% to 15%, 1% to 10%, 1% to 5% or 5% to 30%, 5% to 20%, 5% to 15% fucosylated N-glycans. The fucose-reduced antibodies of the present invention may preferably contain 0%, 1%, 2%, 3%, 4%, 5%, 6%, 7%, 8%, 9%, 10%, 11%, 12%, 13%, 14%, 15%, 16%, 17%, 18%, 19%, 20.0%, 21%, 22%, 23%, 24%, 25%, 26%, 27%, 28%, 29%, 30%, 40%, 41%, 42%, 43%, 44%, 45.0%, 46%, 47%, 48%, 49%, 50%, 51%, 52%, 53%, 54%, 55%, 56%, 57%, 58%, 59%, 60%, 61.0%, 62%, 63%, 64%, 65%, 66%, 67%, 68%, 69%, 70%, 71%, 72%, 73%, 74%, 75%, 76%, 77%, 78%, 79%, or even 80% fucosylated N-glycans. Fucose-reduced antibodies of the present invention may refer to glycosylated antibodies being fucose-reduced. Here, a fucose-reduced antibody of the present invention may refer to a bifunctional monospecific PDL-GEX-Fuc− as well as a trifunctional bispecific PM-PDL-GEX Fuc− antibody

The term “fucose-reduced” refers to the reduction of the content of α-1,6-core fucose, which is attached onto the first N-acetylglucosamine (GlcNac) being part of the mannosyl-chitobiose core (Man3GlcNAc2-Asn), which is bound to each conserved amino acid asparagine N297 in the CH₂ domains of the F_{c} region. This term may also be used interchangeably with the term “de-fucosylated/essentially de-fucosylated” or nouns such as “de-fucosylation” thereof. The term “fucose-reduced” may also be used interchangeably with the term “essentially lacking core-fucosylation”. A fucose-reduced antibody may also be seen in view of the invention as a glyco-optimized antibody.

The term “essentially lacking core-fucosylation” may be used for an antibody, wherein said antibody is fucose-reduced/de-fucosylated or an antibody being glycosylated, having N-linked sugar chains bound to the F_{c} region, wherein among the total complex N-linked sugar chains bound to the F_{c} region, the content of α-1,6-core-fucose may be from 0% to 80%. In other words, the antibody may be from 0% to 80% fucosylated.

The term “core fucosylated N-glycans” refers to N-glycans of a plurality of antibodies, which are core fucosylated. The molar amount of core fucosylated N-glycans relative to the molecular amount of total N-glycans of a plurality of antibodies may be more than 80% or from 0% to 80%. The content of more than 80% core fucosylated N-glycans as it is described for said normal-fucosylated antibodies of the present invention is preferably be determined from a plurality of antibodies, wherein more than 80% of the molecular amount of total N-glycans of a plurality of antibodies may be core α1,6-fucosylated. The content of 0% to 80% core fucosylated N-glycans as it is described for said fucose-reduced antibodies of the present invention may also be determined preferably from a plurality of antibodies, wherein 0% to 80% of molecular amount of N-glycans of a plurality of antibodies may be core α1,6-fucosylated. Core-fucosylation of the N-glycans is determined in Example 1. Fucose addition or reduction may be catalyzed by alpha-(1.6)-fucosyltransferase (FUT8), which is an enzyme that in humans is encoded by the FUT8 gene.

The term “core-fucose” or “core-fucosylated” refers to the monosaccharide fucose, which is attached at position α-1,6 being the first N-acetylglucosamine (GlcNac), which is part of the mannosyl-chitobiose core (Man3GlcNAc2-Asn), which is bound to each conserved amino acid asparagine N297 in the CH₂ domains of the F_{c} region.

The term “content of α-1,6-core-fucose” refers to the amount of core-fucose, which is being attached onto the first N-acetylglucosamine (GlcNac) being part of the mannosyl-chitobiose core (Man3GlcNAc2-Asn), which is bound to each conserved amino acid asparagine N297 in the CH₂ domains of the F_{c} region. Among the total complex N-linked sugar chains bound to the F_{c} region, the content of α-1,6-core-fucose may be more than 80% for the normal-fucosylated antibodies of the present invention or from 0% to 80% for the fucose-reduced antibodies of the present invention. The content of α-1,6-core-fucose may be determined preferably by a plurality of antibodies. Preferably, the content of α-1,6-core-fucose, thus the content of α-1,6-core-fucose of the N-glycans with regard to the plurality of antibodies, may be analyzed by HILIC-UPLC-HiResQToF MSMS (see Example 1).

As it is well known in the art, an “antibody” is an immunoglobulin molecule capable of specific binding to a target (epitope) through at least one epitope recognition site, located in the variable region of the immunoglobulin molecule. The term “antibody” as used herein may comprise monoclonal and polyclonal antibodies, as well as (naturally occurring or synthetic) fragments or variants thereof, including fusion proteins comprising an antibody portion with an antigen-binding fragment of the required specificity and any other modified configuration of the antibody that comprises an antigen-binding site or fragment (epitope recognition site) of the required specificity. Illustrative examples of the antibody fragments or antibodies may include dAb, F_{ab}, F_{ab}′, F(ab′)₂, Fᵥ, single chain Fᵥs (scFᵥ), single chain Fᵥs (scFᵥs) coupled to the constant domain of the kappa light chains or to the CH₃ domain of the heavy chains, diabodies, and minibodies. The antibody of the present invention when referred to herein may also be a composition comprising a plurality of antibodies.

An antibody is composed of two heavy (H) and two light (L) chains connected by disulfide bonds. They are being separated functionally into a F_{ab} (fragment, antigen-binding) region capable of binding to antigens and into a F_{c} (fragment, crystallizable) region that specifies effector functions such as activation of complement or binding to F_{c} receptors.

The term “plurality of antibodies” refers to the amount of antibodies which is preferably required for glycan analysis, preferably 15 μg.

The antibody of the present invention may be a humanized antibody (or antigen-binding variant or fragment thereof). The term “humanized antibody” refers to an antibody containing a minimal sequence derived from a non-human antibody. In general, humanized antibodies are human immunoglobulins comprising residues from a hypervariable region of an immunoglobulin derived from non-human species such as mouse, rat, rabbit or non-human primate (“donor antibody”) grafted onto the human immunoglobulin (“recipient antibody”). In some instances, frame work region (FR) residues of the human immunoglobulin are replaced by corresponding non-human residues. Furthermore, humanized antibodies may comprise residues that are neither found in the recipient antibody nor in the donor antibody. These modifications are made to further refine antibody performance. In general, the humanized antibody may comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also may comprise at least a portion of an immunoglobulin constant region (F_{c}), typically that of a human immunoglobulin.

The antibody may be a monospecific antibody. The term “monospecific” refers to any homogeneous antibody or antigen-binding region thereof which is reactive with, preferably specifically reactive with, a single epitope or antigenic determinant. Antibodies that all have affinity for the same antigen; antibodies that are specific to one antigen or one epitope; or antibodies specific to one type of cell or tissue may all refer to “monospecific antibodies”. The term “monospecific antibody” may also refer to a monoclonal antibody, also abbreviated “MoAb”, as that term is conventionally understood. But monospecific antibodies may also be produced by other means than producing them from a common germ cell as it is done for monoclonal antibodies. The term “monospecific antibody” as used herein may, however, refers to homogeneous antibodies which are native, modified, or synthetic, and can include hybrid or chimeric antibodies. In particular, a monospecific antibody of the present invention preferably comprises V_{H} and V_{L} domains binding to an immune checkpoint protein, preferably said immune checkpoint protein is PD-L1. Thus, a monospecific antibody of the present invention may include a PD-L1 antibody. The present invention may further envisage an antibody comprising V_{H} and V_{L} domains binding to a cancer antigen, preferably said cancer antigen is TA-MUC1. Thus, a monospecific antibody of the present invention may also include a TA-MUC1 antibody.

If a monospecific antibody binding to PD-L1 is referred to in the present invention, said antibody has the amino acid sequence shown in SEQ ID NO. 40 and 50. Here, SEQ ID NO. 40 refers to the heavy chain of said PD-L1 antibody, whereas SEQ ID NO. 50 refers to the light chain of said PD-L1 antibody. The present invention may also comprise an antibody binding to PD-L1 comprising polypeptide chains, wherein each of the polypeptide chain may have at least 50% sequence identity to any one of SEQ ID NO. 40 and 50. An antibody binding to PD-L1 may comprise polypeptide chains, wherein each of the polypeptide chain may have at least 50%, 55%, 60%, 65%, 70%, 75%, 80%, 85%, 90%, 95%, 96%, 97%, 98%, or at least 99% sequence identity to any one of SEQ ID NO. 40 and 50. The present invention may envisage an antibody binding to PD-L1 comprising a heavy chain capable of binding to PD-L1, having at least 50%, 55%, 60%, 65%, 70%, 75%, 80%, 85%, 90%, 95%, 96%, 97%, 98%, or at least 99% sequence identity to SEQ ID NO. 40 and a light chain having at least 50%, 55%, 60%, 65%, 70%, 75%, 80%, 85%, 90%, 95%, 96%, 97%, 98%, or at least 99% sequence identity to SEQ ID NO. 50. Further, the present invention may also comprise an antibody binding to PD-L1 having any one of the amino acid sequences shown in SEQ ID NOs. 41-49 and SEQ ID NO. 50. Herein, SEQ ID NOs. 41-49 refer to the mutated heavy chains of the antibody binding to PD-L1 of the present invention having different mutations in the CDRs of the V_{H} domain of said antibody.

The present invention may also comprise an antibody binding to PD-L1 having different mutations in the CDRs of the V_{H} domain of said antibody having the amino acid sequences as shown in SEQ ID NOs. 51-59 and 18. Herein, the SEQ ID NOs. 51-59 refer to the mutated V_{H} domains of the antibody binding to PD-L1 of the present invention having different mutations in the CDRs of the V_{H} domain of said antibody.

An antibody of the present invention having different mutations in the CDRs of the V_{H} domain of said antibody may comprise the following V_{H} CDRs having the amino acid sequences as shown in SEQ ID No. 60 and 68, which preferably confer binding to PD-L1, or having the amino acid sequences as shown in SEQ ID NO. 62 and 69, which preferably confer binding to PD-L1, or having the amino acid sequences as shown in SEQ ID NO. 63 and 70, which preferably confer binding to PD-L1, or having the amino acid sequence as shown in SEQ ID NO. 64, which preferably confer binding to PD-L1, or having the amino acid sequences as shown in SEQ ID NO. 65 and 71, which preferably confer binding to PD-L1, or having the amino acid sequences as shown in SEQ ID NO. 66 and 72, which preferably confer binding to PD-L1, or having the amino acid sequences as shown in SEQ ID NO. 63 and 72, which preferably confer binding to PD-L1, or having the amino acid sequences as shown in SEQ ID NO. 67 and 74, which preferably confer binding to PD-L1, or having the amino acid sequences as shown in SEQ ID NO. 63 and 68, which preferably confer binding to PD-L1, or having the amino acid sequence as shown in SEQ ID NO. 61, which preferably confer binding to PD-L1, or having the amino acid sequence as shown in SEQ ID NO. 73, which preferably confer binding to PD-L1, or having the amino acid sequence as shown in SEQ ID NO. 75, which preferably confer binding to PD-L1.

The term “bispecific antibody” may in the context of the present invention to be understood as an antibody with two different antigen-binding regions (based on sequence information). This can mean different target binding but includes as well binding to different epitopes in one target. In particular, a bispecific antibody of the present invention is preferably capable of binding to TA-MUC1 and further being capable of binding to an immune checkpoint protein, wherein said immune checkpoint protein is preferably PD-L1. Further, the present invention may also provide an antibody preferably being capable of binding to PD-L1 and further being capable of binding to a cancer antigen, wherein said cancer antigen is preferably TA-MUC1. The present invention may also contemplate an anti-PD-L1 antibody further binding to another molecule on immune cells, thus having an antibody being capable of binding to PD-L1 and further being capable of binding to another molecule on immune cells.

The present invention usually envisage a bispecific antibody binding to TA-MUC1 and further binding to PD-L1 having the amino acid sequence shown in SEQ ID NO. 13 (or SEQ ID NO. 37) and 14 and/or SEQ ID No. 15 and 16 (or SEQ ID NO. 38). Here, SEQ ID No. 13 (or SEQ ID NO. 37) refers to the light chain, wherein a scFᵥ region binding to PD-L1 is coupled to the constant domain of said light chain, whereas SEQ ID No. 14 refers to the heavy chain of the antibody. SEQ ID No. 15 refers to the heavy chain, wherein a scFᵥ region binding to PD-L1 is coupled to the CH₃ domain of the F_{c} region, whereas SEQ ID No. 16 (or SEQ ID NO. 38) refers to the light chain of the antibody. The bispecific antibody comprising a light chain coupled to a scFᵥ region (SEQ ID No. 13 or SEQ ID NO. 37), wherein the scFᵥ region is coupled to the constant domain of said light chain and being capable of binding to PD-L1, and a heavy chain (SEQ ID No. 14) may be preferred in the present invention. The present invention may also comprise an antibody with two light chains coupled to scFᵥ regions being capable of binding to PD-L1 according to SEQ ID No. 13 (or SEQ ID NO. 37) and two heavy chains according to SEQ ID No. 14.

The present invention may also comprise an antibody comprising polypeptide chains, wherein each of the polypeptide chain may have at least 50% sequence identity to any one of SEQ ID No. 13 (or SEQ ID NO. 37) and 14 as well as 15 and 16 (or SEQ ID NO. 38). An antibody of the present invention may comprise polypeptide chains, wherein each of the polypeptide chain may have at least 50%, 55%, 60%, 65%, 70%, 75%, 80%, 85%, 90%, 95%, 96%, 97%, 98%, or at least 99% sequence identity to any one of SEQ ID No. 13 (or SEQ ID NO. 37) and 14 as well as 15 and 16 (or SEQ ID NO. 38). The present invention may envisage an antibody comprising a light chain coupled to a scFᵥ region capable of binding to PD-L1, having at least 50%, 55%, 60%, 65%, 70%, 75%, 80%, 85%, 90%, 95%, 96%, 97%, 98%, or at least 99% sequence identity to SEQ ID No. 13 (or SEQ ID NO. 37) and a heavy chain having at least 50%, 55%, 60%, 65%, 70%, 75%, 80%, 85%, 90%, 95%, 96%, 97%, 98%, or at least 99% sequence identity to SEQ ID NO. 14. The present invention may further contemplate an antibody with two light chains coupled to scFᵥ regions capable of binding to PD-L1 having at least 50%, 55%, 60%, 65%, 70%, 75%, 80%, 85%, 90%, 95%, 96%, 97%, 98%, or at least 99% sequence identity to SEQ ID NO. 13 (or SEQ ID NO. 37) and two heavy chains having at least 50%, 55%, 60%, 65%, 70%, 75%, 80%, 85%, 90%, 95%, 96%, 97%, 98%, or at least 99% sequence identity to SEQ ID NO. 14. The present invention may also include an antibody comprising a heavy chain coupled to a scFᵥ region capable of binding to PD-L1 having at least 50%, 55%, 60%, 65%, 70%, 75%, 80%, 85%, 90%, 95%, 96%, 97%, 98%, or at least 99% sequence identity to SEQ ID No. 15 and a light chain having at least 50%, 55%, 60%, 65%, 70%, 75%, 80%, 85%, 90%, 95%, 96%, 97%, 98%, or at least 99% sequence identity to SEQ ID NO. 16 (or SEQ ID NO. 38). The present invention may further contemplate an antibody with two heavy chains coupled to scFᵥ regions capable of binding to PD-L1 having at least 50%, 55%, 60%, 65%, 70%, 75%, 80%, 85%, 90%, 95%, 96%, 97%, 98%, or at least 99% sequence identity to SEQ ID NO. 15 and two light chains having at least 50%, 55%, 60%, 65%, 70%, 75%, 80%, 85%, 90%, 95%, 96%, 97%, 98%, or at least 99% sequence identity to SEQ ID NO. 16 (or SEQ ID NO. 38). An antibody of the present invention comprising polypeptide chains, wherein each of the polypeptide chain may have at least 50%, 55%, 60%, 65%, 70%, 75%, 80%, 85%, 90%, 95%, 96%, 97%, 98%, or at least 99% sequence identity to any one of SEQ ID No. 13 (or SEQ ID NO. 37) and 14 as well as 15 and 16 (or SEQ ID NO. 38) may also be capable of binding to PD-L1 and TA-MUC1.

If a bispecific antibody binding to TA-MUC1 and binding to PD-L1 with its scFᵥ region is addressed in the present invention having different mutations in the CDRs of the V_{H} domain of the scFᵥ region, said antibody may also have any one of the amino acid sequences as shown in SEQ ID NOs. 76-79 and 14. Herein, SEQ ID NOs. 76-79 refer to the light chain, wherein a scFᵥ region binding to PD-L1 is coupled to the constant domain of said light chain of the bispecific antibody binding to TA-MUC1 and binding to PD-L1 with its scFᵥ region, which comprises different mutations in the CDRs of the V_{H} domain of the scFᵥ region binding to PD-L1. Preferably, said bispecific antibody binding to TA-MUC1 and binding to PD-L1 with its scFᵥ region having different mutations in the CDRs of the V_{H} domain of the scFᵥ region, has the amino acid sequences as shown in SEQ ID NO. 77 or 78.

Also comprised by the present invention is a bispecific antibody binding to TA-MUC1 and binding to PD-L1 with its scFᵥ region having different mutations in the CDRs of the V_{H} domain of the scFᵥ region, wherein said antibody may also have any one of the amino acid sequences as shown in SEQ ID NOs. 80-83 and 16 (or SEQ ID NO. 38). Herein, SEQ ID NOs. 80-83 refer to the heavy chain, wherein a scFᵥ region binding to PD-L1 is coupled to the CH₃ domain of the F_{c} region of the bispecific antibody binding to TA-MUC1 and binding to PD-L1 with its scFᵥ region, which comprises different mutations in the CDRs of the V_{H} domain of the scFᵥ region binding to PD-L1.

The term “non-mutated antibody” refers to an antibody, which may not comprise one or more sequence mutations selected from S238D, S239D, 1332E, A330L, S298A, E333A, L334A, G236A, L235V, F243L, R292P, Y300L, V3051, and P396L according to EU-nomenclature. Preferably, a non-mutated antibody may not comprise the triple mutation G236A/S239D/1332E and the quintuple mutation L235V/F243L/R292P/Y300L/P396L

The term “F_{ab} region” refers to the fragment, antigen-binding region consisting one complete light chain and the variable and C_{H}1 domain of one heavy chain. However, the F_{ab} region can also be divided into the variable fragment (Fᵥ) composed of the V_{H} and V_{L} domains, and a constant fragment (F_{b}) composed of the constant domain of the light chain (C_{L}) and the C_{H}1 domain.

The term “F_{c} region” refers to the fragment, crystallizable region consisting of the second constant domains (CH₂) and the third constant domains (CH₃) of the antibody's two heavy chains. It specifies effector functions such as activation of complement or binding to F_{c} receptors.

The term “scFᵥ region” refers to the term single-chain fragment variable region comprising a variable domain of the heavy chain (V_{H} domain) and a variable domain of the light chain (V_{L} domain). scFᵥ regions may be coupled symmetrically to the constant domain of the light chain (“C-terminal-fusion”) of said antibody or to the CH₃ domain of the F_{c} region of said antibody (“C-terminal-fusion”) by linkers, preferably by GS-linkers. ScFᵥ regions are coupled by linkers either to the constant domain of the light chain or to the CH₃ domain of the F_{c} region of said antibody. The linker may in principle have any number of amino acids and any amino acid sequence. The linker may comprise at least 3, 5, 8, 10, 15 or 20 amino acids, preferably at least 5 amino acids. Further, the linker may comprise less than 50 or less than 40, 35, 30, 25, 20 amino acids, preferably less than 45 amino acids. In particular, the linker may comprise from 5 to 20 amino acids, preferably 5 amino acids. Preferably, the linker may consist of glycine and serine residues. Glycine and serine may be present in the linker in a ratio of 2 to 1, 3 to 1, 4 to 1 or 5 to 1 (number of glycine residues to number of serine residues). For example, the linker may comprise a sequence of four glycine residues followed by one serine residue, and in particular 1, 2, 3, 4, 5 or 6 repeats of this sequence. Linkers consisting of 2 repeats of the amino acid sequence may refer to (GGGGS)₂, 4 repeats of the amino acid sequence may refer to (GGGGS)₄ and 6 repeats of the amino acid sequence refer to (GGGGS)₆. Especially, linkers consisting of 4 repeats of the amino acid sequence (GGGGS)₄ may be preferred. The linker, which couples scFᵥ regions to the constant domain of the light chain or to the CH₃ domain of the heavy chain may be a GS-linker. Additionally, the linker may comprise sequences which show no or only minor immunogenic potential in humans, preferably sequences which are human sequences or naturally occurring sequences. Consequently, the linkers and the adjacent amino acids may show no or only minor immunogenic potential.”

Further, a scFᵥ region preferably consists of one V_{H} (SEQ ID No. 17) and one V_{L} domain (SEQ ID No. 18), connected by GS-linkers, preferably by a 4 GS-linker. An antibody of the invention may have two scFᵥ regions, both either coupled to the constant domain of the light chains of said antibody or to the CH₃ domain of the F_{c} region of said antibody. Also comprised by the present invention may be a scFᵥ region consisting of one mutated V_{H} domain, preferably having any one of amino acid sequences as shown in SEQ ID NOs. 51-59 and of one non-mutated V_{L} domain as shown in SEQ ID No. 18, if a bispecific antibody binding to TA-MUC1 and binding to PD-L1 with its scFᵥ region, which comprises different mutations in the CDRs of the V_{H} domain of the scFᵥ region binding to PD-L1, is addressed in the present invention.

ScFᵥ regions may be genetically engineered, but unmodified sequences may also be used to form scFᵥ regions. ScFᵥ regions recapitulate the monovalent antigen binding characteristics of the original, parent antibody, despite removal of the constant regions.

Said antibody of the present invention may comprise single chain Fᵥ regions binding to an immune checkpoint protein, wherein said immune checkpoint protein is preferably PD-L1. Those single chain Fᵥ regions may be coupled to the constant domain of the light chain or to the CH₃ domain of the F_{c} region. An antibody of the present invention may comprise the following V_{H} and V_{L} domain CDRs having the amino acid sequence shown in SEQ ID Nos. 1-6, which preferably confer binding to PD-L1. SEQ ID Nos. 1-3 may refer to the V_{H} domain CDRs of the scFᵥ regions, whereas SEQ ID Nos. 4-6 may refer to the V_{L} domain CDRs of the scFᵥ regions:

SEQ ID No. 1: Gly Phe Thr Phe Ser Asp Ser Trp Ile His (CDR1 in the V_{H} domain of the PD-L1 binding site)
SEQ ID No. 2: Ala Trp Ile Ser Pro Tyr Gly Gly Ser Thr Tyr Tyr Ala Asp Ser Val Lys Gly (CDR2 in the V_{H} domain of the PD-L1 binding site),
SEQ ID No. 3: Arg His Trp Pro Gly Gly Phe Asp Tyr (CDR3 in the V_{H} domain of the PD-L1 binding site).
SEQ ID No. 4: Arg Ala Ser Gln Asp Val Ser Thr Ala Val Ala (CDR1 in the V_{L} domain of the PD-L1 binding site),
SEQ ID No. 5: Ser Ala Ser Phe Leu Tyr Ser (CDR2 in the V_{L} domain of the PD-L1 binding site),
SEQ ID No. 6: Gln Gln Tyr Leu Tyr His Pro Ala Thr (CD3 in the V_{L} domain of the PD-L1 binding site).

The present invention may also comprise an antibody, wherein the V_{H} domain CDR1 of the scFᵥ region capable of binding to PD-L1 may have 1, 2, 3, 4, or 5 mutations as compared to SEQ ID No. 1. Further, the present invention may comprise an antibody, wherein the V_{H} domain CDR2 of the scFᵥ region capable of binding to PD-L1 may have 1, 2, 3, 4, 5, 6, 7, 8, or 9 mutations as compared to SEQ ID No. 2. Additionally, the invention may contemplate an antibody, wherein the V_{H} domain CDR3 of the scFᵥ region capable of binding to PD-L1 may have 1, 2, 3, 4, or 5 mutations as compared to SEQ ID No. 3. Further, the present invention may envisage an antibody, wherein the V_{H} domain frame work region 1 of the scFᵥ region may have 1, 2, 3, 4, 5, 6, 7, 8, 9, 10, 11 or 12 mutations compared to frame work region 1 of SEQ ID No. 21. Further, the present invention may envisage an antibody, wherein the V_{H} domain frame work region 2 of the scFᵥ region may have 1, 2, 3, 4, 5 or 6 mutations compared to frame work region 2 of SEQ ID No. 22. Additionally, the present invention may envisage an antibody, wherein the V_{H} domain frame work region 3 of the scFᵥ region may have 1, 2, 3, 4, 5, 6, 7, 8, 9, 10, 11, 12, 13, 14, 15, or 16 mutations compared to frame work region 3 of SEQ ID No. 23. The present invention may envisage an antibody, wherein the V_{H} domain frame work region 4 of the scFᵥ region may have 1, 2, 3, 4, or 5 mutations compared to frame work region 4 of SEQ ID No. 24. The present invention may also envisage an antibody, wherein the V_{L} domain CDR1 of the scFᵥ region capable of binding to PD-L1 may have 1, 2, 3, 4, or 5 mutations as compared to SEQ ID No. 4. The present invention may include an antibody having 1, 2, or 3 mutations in the V_{L} domain CDR2 of the scFᵥ region capable of binding to PD-L1 as compared to SEQ ID No. 5. The present invention may also encompass an antibody having 1, 2, 3, or 4 mutations in the V_{L}domain CDR3 of the scFᵥ region as compared to SEQ ID No. 6. Further, the present invention may envisage an antibody, wherein the V_{L} domain frame work region 1 of the scFᵥ region may have 1, 2, 3, 4, 5, 6, 7, 8, 9, 10, or 11 mutations compared to frame work region 1 of SEQ ID No. 25. Further, the present invention may envisage an antibody, wherein the V_{L} domain frame work region 2 of the scFᵥ region may have 1, 2, 3, 4, 5, 6, or 7 mutations compared to frame work region 2 of SEQ ID No. 26. Additionally, the present invention may envisage an antibody, wherein the V_{L} domain frame work region 3 of the scFᵥ region may have 1, 2, 3, 4, 5, 6, 7, 8, 9, 10, 11, 12, 13, 14, 15, or 16 mutations compared to frame work region 3 of SEQ ID No. 27. The present invention may envisage an antibody, wherein the V_{L} domain frame work region 4 of the scFᵥ region may have 1, 2, 3, 4, or 5 mutations compared to frame work region 4 of SEQ ID No. 28. An antibody of the present invention having one or more V_{H} and V_{L} domain CDRs having said mutations, may also confer binding to PD-L1. Additionally, the present invention may also contemplate an antibody comprising V_{H} and V_{L} domain CDRs of scFᵥ regions, which may be capable of binding a cancer antigen, preferably TA-MUC1.

If a bispecific antibody binding to TA-MUC1 and binding to PD-L1 with its scFᵥ region having different mutations in the CDRs of the V_{H} domain of the scFᵥ region is addressed in the present invention, said antibody may preferably comprise the following V_{H} CDRs which preferably confer binding to PD-L1: SEQ ID NO. 64 having a mutation of glycine to alanine at position 26 in the CDR1 of the V_{H} domain according to Kabat-numbering and having a mutation of aspartic acid to glutamic acid at position 31 in the CDR1 of the V_{H} domain according to Kabat-numbering

or SEQ ID NO. 66 having a mutation of threonine to serine at position 28 in the CDR1 of the V_{H} domain according to Kabat-numbering and SEQ ID NO. 72 having a mutation of serine to threonine at position 62 according to Kabat-numbering in the CDR2 of the V_{H} domain as indicated elsewhere herein.

The term “V_{H} and V_{L} domain” may refer to the variable domain of the heavy chain and the variable domain of the light chain of the F_{ab} region of an antibody of the present invention. Is the variable domain of the heavy chain and the variable domain of the light chain of the scFᵥ region addressed in the present invention, the term “V_{H} and V_{L} domain of the scFᵥ region” may be used.

Said V_{H} (SEQ ID No. 19) and V_{L} domains (SEQ ID No. 20 or SEQ ID NO. 39) of the antibody of the present invention may be capable of binding to a cancer antigen, wherein said cancer antigen is preferably TA-MUC1. Thus, a bispecific antibody of the present invention may comprise V_{H} and V_{L} domains preferably binding to TA-MUC1. An antibody of the present invention may comprise the following V_{H} and V_{L} domain CDRs having the amino acid sequence shown in SEQ ID Nos. 7-12, which preferably confer binding to TA-MUC1. SEQ ID Nos. 7-9 may refer to the V_{H} domain CDRs, whereas SEQ ID Nos. 10-12 may refer to the V_{L} domain CDRs:

SEQ ID No. 7: Asn Tyr Trp Met Asn (CDR1 in the V_{H} domain of the TA-MUC1 binding site),
SEQ ID No. 8: Glu Ile Arg Leu Lys Ser Asn Asn Tyr Thr Thr His Tyr Ala Glu Ser Val Lys Gly (CDR2 in the V_{H} domain of the TA-MUC1 binding site),
SEQ ID No. 9: His Tyr Tyr Phe Asp Tyr (CDR3 in the V_{H} domain of the TA-MUC1 binding site).
SEQ ID No. 10: Arg Ser Ser Lys Ser Leu Leu His Ser Asn Gly Ile Thr Tyr Phe Phe (CDR1 in the V_{L} domain of the TA-MUC1 binding site),
SEQ ID No. 11: Gln Met Ser Asn Leu Ala Ser (CDR2 in the V_{L} domain of the TA-MUC1 binding site),
SEQ ID No. 12: Ala Gln Asn Leu Glu Leu Pro Pro Thr (CDR3 in the V_{L} domain of the TA-MUC1 binding site).

The present invention may also comprise an antibody, wherein the V_{H} domain CDR1 region may have 1, 2, or 3 mutations as compared to SEQ ID No. 7. Further, the present invention may comprise an antibody, wherein the V_{H} domain CDR2 may have 1, 2, 3, 4, 5, 6, 7, 8, or 9 mutations as compared to SEQ ID No. 8. Additionally, the invention may contemplate an antibody, wherein the V_{H} domain CDR3 may have 1, 2, or 3 mutations as compared to SEQ ID No. 9. Further, the present invention may envisage an antibody, wherein the V_{H} domain frame work region 1 may have 1, 2, 3, 4, 5, 6, 7, 8, 9, 10, 11, 12, 13, 14, or 15 mutations compared to frame work region 1 of SEQ ID No. 29. Further, the present invention may envisage an antibody, wherein the V_{H} domain frame work region 2 may have 1, 2, 3, 4, 5, 6, or 7 mutations compared to frame work region 2 of SEQ ID No. 30. Additionally, the present invention may envisage an antibody, wherein the V_{H} domain frame work region 3 may have 1, 2, 3, 4, 5, 6, 7, 8, 9, 10, 11, 12, 13, 14, 15, or 16 mutations compared to frame work region 3 of SEQ ID No. 31 The present invention may envisage an antibody, wherein the V_{H} domain frame work region 4 may have 1, 2, 3, 4, or 5 mutations compared to frame work region 4 of SEQ ID No. 32.

The present invention may also envisage an antibody, wherein the V_{L} domain CDR1 may have 1, 2, 3, 4, 5, 6, 7, or 8 mutations as compared to SEQ ID No. 10. The present invention may include an antibody having 1, 2, or 3 mutations in the V_{L} domain CDR2 as compared to SEQ ID No. 11. The present invention may also encompass an antibody having 1, 2, 3, or 4 mutations in the V_{L} domain CDR3 as compared to SEQ ID No. 12. Further, the present invention may envisage an antibody, wherein the V_{L} domain frame work region 1 may have 1, 2, 3, 4, 5, 6, 7, 8, 9, 10, or 11 mutations compared to frame work region 1 of SEQ ID No. 33. Further, the present invention may envisage an antibody, wherein the V_{L} domain frame work region 2 may have 1, 2, 3, 4, 5, 6, or 7 mutations compared to frame work region 2 of SEQ ID No. 34. Additionally, the present invention may envisage an antibody, wherein the V_{L} domain frame work region 3 may have 1, 2, 3, 4, 5, 6, 7, 8, 9, 10, 11, 12, 13, 14, 15, or 16 mutations compared to frame work region 3 of SEQ ID No. 35. The present invention may envisage an antibody, wherein the V_{L} domain frame work region 4 may have 1, 2, 3, 4, 5, or 6 mutations compared to frame work region 4 of SEQ ID No. 36.

Further, an antibody of the present invention having one or more V_{H} and V_{L} domain CDRs having said mutations, may also confer binding to TA-MUC1. The present invention may also contemplate an antibody comprising V_{H} and V_{L} domain CDRs, which may be capable of binding an immune checkpoint protein, preferably PD-L1.

The term “frame work region” refers to the amino acid region before and after a CDR and inbetween CDRs either in the V_{H} and V_{L} domain or in the V_{H} and V_{L} domain of the scFᵥ regions.

The term “CDRs” refers to complementarity-determining regions, which refer to variable loops of β-strands, three each on the variable domains of the light (V_{L}) and heavy (V_{H}) chains in immunoglobulins (antibodies) generated by B-cells respectively or in single chain Fᵥ regions coupled to an immunoglobulin being responsible for binding to the antigen. Unless otherwise indicated CDRs sequences of the disclosure follow the definition by Maass 2007 (Journal of Immunological Methods 324 (2007) 13-25). Other standards for defining CDRs exist as well, such as the definition according to Kabat CDRs, as described in Sequences of Proteins of immunological Interest, US Department of Health and Human Services (1991), eds. Kabat et al. Another standard for characterizing the antigen binding site is to refer to the hypervariable loops as described by Chothia (see, e.g., Chothia, et al. (1992); J. Mol. Biol. 227:799-817; and Tomlinson et al. (1995) EMBO J. 14:4628-4638). Still another standard is the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). It is understood that embodiments described with respect to the CDR definition of Maass, can alternatively be implemented using similar described relationships such as with respect to Kabat CDRs, Chothia hypervariable loops or to the AbM-defined loops.

The term “mutation” refers to substitution, insertion and/or deletion. Mutations may occur in the V_{H} and V_{L} domain CDRs and/or in the corresponding frame work region of the V_{H} and V_{L} domains. Mutations may also occur in the V_{H} and V_{L} domain CDRs of the scFᵥ regions and/or in the corresponding frame work region of the V_{H} and V_{L} domains of the scFᵥ regions.

The term “GS-linker” refers to a peptide linker or a sequence with stretches of glycine (Gly/G) and serine (Ser/S) residues. A GS-linker may contain 5, 10, 15, 20, 25 or more than 25 amino acids, preferably 5 amino acids. Mostly, the common (G4S) 4 linker repeat (here called as 4 GS-linker—“GGGGS-GGGGS-GGGGS-GGGGS”) or the (G4S) 6 linker peptide (here called as 6 GS-linker—“GGGGS-GGGGS-GGGGS-GGGGS-GGGGS-GGGGS”) may be used in an antibody. In general, a 4 GS-linker may couple either the V_{H}-domain of the scFᵥ region to the constant domain of the light chain or the V_{H}-domain of the scFᵥ region to the CH₃ domain of the F_{c} region of said antibody. A 6 GS-linker may couple the V_{H}-domain to the V_{L}-domain of the scFᵥ region, having a V_{H}-linker-V_{L} orientation. Here, the bispecific normal-fucosylated and the bispecific fucose-reduced antibodies of the present invention may comprise 4 GS-linkers. The first 4 GS-linker may couple the V_{H}-domain of the scFᵥ region either to the constant domain of the light chain or to the CH₃ domain of the F_{c} region of said antibodies, the other 4 GS-linker may couple the V_{H}-domain to the V_{L}-domain of the scFᵥ region, having a V_{H}-linker-V_{L} orientation.

The term “bifunctional monospecific antibody” may refer to an antibody of the present invention, wherein the F_{c} region may bind to an FcyR receptor, preferably to FcyRIIIa and the V_{H} and V_{L} domains may bind to an immune checkpoint protein, preferably said immune checkpoint protein is PD-L1. The present invention may also comprises an antibody comprising a F_{c} region binding to an FcyR receptor, preferably to FcyRIIIa and the V_{H} and V_{L} domains binding to a cancer antigen, preferably said cancer antigen is TA-MUC1.

The term “trifunctional bispecific antibody” may refer to an antibody of the present invention, wherein the F_{c} region may bind to an FcyR receptor, preferably to FcyRIIIa and the V_{H} and V_{L} domains may bind to a cancer antigen, preferably said cancer antigen is TA-MUC1. Further, said trifunctional bispecific antibody capable of binding to TA-MUC1 may further have single chain Fᵥ regions, which may bind to an immune checkpoint protein, preferably said immune checkpoint protein is PD-L1. Said trifunctional bispecific antibody capable of binding to TA-MUC1 and with its scFᵥ regions capable of binding to PD-L1 may be preferred by the present invention. The term “trifunctional bispecific antibody” may also refer to an antibody of the present invention, wherein the F_{c} region may bind to an FcyR receptor, preferably to FcyRIIIa and the V_{H} and V_{L} domains may bind to an immune checkpoint protein, preferably said immune checkpoint protein is PD-L1. Further, the trifunctional bispecific antibody capable of binding to PD-L1 may further have single chain Fᵥ regions, which may bind to a cancer antigen, preferably said cancer antigen is TA-MUC1.

The term “PM-PDL-GEX” refers to a PankoMab antibody combined with PD-L1 specificity, also called a bispecific PankoMab-antiPDL1-GEX antibody or anti-PD-L1/TA-MUC1 hIgG1 antibody. A PM-PDL-GEX antibody is developed by Glycotope GmbH. Here, the PankoMab antibody with PD-L1 specificity is trifunctional bispecific. Further, the anti-PD-L1 part as a scFᵥ region of the PankoMab-anti-PD-L1-GEX antibody may comprise an antagonistic effect.

The term “PankoMab” refers to a humanized monoclonal antibody recognizing the tumor-specific epitope of mucin-1 (TA-MUC1), enabling it to differentiate between tumor MUC1 and non-tumor MUC1 epitopes. It is developed by Glycotope GmbH. A PankoMab antibody of the present invention is capable of binding to a cancer antigen, preferably TA-MUC1 and is combined with PD-L1 specificity, thus being capable of binding with its scFᵥ regions to an immune checkpoint protein, preferably PD-L1.

The term “glyco-optimized antibody” refers to an antibody, whose glycosylation of the oligosaccharides in its F_{c} region is modified. Here, the term “glyco-optimized” refers to a de-fucosylation of the oligosaccharide structure at the α-1,6-position. Glyco-optimization offers the opportunity to further increase the anti-tumor T cell response due to increased binding to FcyRIIIs, preferably to FcyRIIIa. Thus, a glyco-optimized antibody has the potential to directly kill tumor cells and deplete PD-L1 immunosuppressive cells due to FcyR-bearing immune cells.

The term “immune checkpoint protein” refers to a protein molecule in the immune system, which modulates immune response, either anti-inflammatory or pro-inflammatory. They monitor the correct function of the immune response by either turning up a signal (co-stimulatory molecules) or turning down a signal. There are inhibitory (anti-inflammatory) immune checkpoint proteins such as A2AR, B7-H3 (CD276), B7-H4 (VTCN1), BTLA, CTLA-4, IDO, KIR, LAG3, PD-1, PD-L1, TIM-3, VISTA (protein) and pro-inflammatory immune checkpoint proteins such as CD27, CD40, OX40, GITR and CD137 (4-1BB). The present invention may prefer the inhibitory immune checkpoint proteins. Here, the immune checkpoint protein preferably refers to PD-L1.

The term “cancer antigen” refers to an antigenic substance produced in cancer cells. Cancer antigens, due to their relative abundance in cancer cells are useful in identifying specific cancer cells. Certain cancers have certain cancer antigens in abundance. Cancer-associated antigens may include, but are not limited to HER2, EGFR, VEGF, TA-MUC1, PSA. Here, the cancer antigen preferably refers to TA-MUC1. The term “tumor antigen” can be used interchangeably.

The term “derived from” or “derived therefrom” may be used interchangeably with the term “originated from”/“originated therefrom” or “obtained from”/“obtained therefrom”. For example, a cell or cell line may originate from another cell or a cell line mentioned in the present invention.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number. For example, less than 20 means less than the number indicated. Similarly, more than or greater than means more than or greater than the indicated number, f.e. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be had from the following examples, offered for illustrative purposes only. The examples are not intended to limit the scope of the present invention in any way.

### EXAMPLES

Hereinafter, the present invention is described in more detail and specifically with reference to the Examples, which however are not intended to limit the present invention.

### Example 1: The Monospecific PDL-GEX Fuc− and Bispecific PM-PDL-GEX Fuc− have Reduced Core Fucosylation Compared to the Monospecific PDL-GEX H9D8 and Bispecific PM-PDL-GEX H9D8

The monospecific PDL-GEX Fuc− and the bispecific PM-PDL-GEX Fuc− contain only low percentages of core fucosylated N-glycans and are therefore referred as fucose-reduced (FIG. 1).

It is discussed in the literature that F_{c} N-glycosylation predominantly influences binding of antibodies to the Fc receptor and therefore play role for mediating ADCC. N-glycosylation of monospecific antibodies PDL-GEX H9D8 and PDL-GEX Fuc− and of bispecific antibodies PM-PDL-GEX H9D8 and PM-PDL-GEX Fuc− was analyzed by HILIC-UPLC-HiResQToF MSMS (hydrophilic interaction ultra-performance chromatography coupled to high resolution quadrupole time-of-flight tandem mass spectrometry).

Briefly, the antibody was denatured by RapiGest SF® (Waters Inc.) and tris-(2-carboxyethyl)phosphine (120 min, 95° C.). N-Glycans were released by Rapid PNGase F® (10 min, 55° C.; Waters Inc.), followed by fluorescence tagging with RapiFluor MS® reagent in dimethylformamide for 5 min at room temperature. For clean-up of tagged glycans a pElution Plate (HILIC SPE) was used. Labeled N-glycans were separated on a HILIC phase (UPLC BEH GLYCAN 1.7 150 mm, Waters Inc.) employing an ultra-performance chromatography device (I-Class, Waters Inc.) including a fluorescence detector. RapiGest SF® tagged N-glycans were detected at 265 nm excitation wavelength and 425 nm emission wavelength. Fluorescence signals were employed for glycan quantification. In series to the fluorescence detector a high resolution mass spectrometer was coupled (Impact HD, Bruker Daltonik GmbH). Precursor in combination with a series of fragment masses allowed for unambiguous identification of glycan structures.

### Example 2: A Fucose-Reduced Anti-PD-L1 hIgG1 and a Fucose-Reduced Bispecific Anti-PD-L1/TA-MUC1 hIgG1 Show Comparable Blocking Capacity Compared to their Normal-Fucosylated Counterparts

A fucose-reduced anti-PD-L1 hIgG1 and a fucose-reduced bispecific anti-PD-L1/TA-MUC1 hIgG1 show comparable blocking capacity for PD-L1/PD-1 and PD-L1/CD80 blocking.

Two different competitive ELISAs were developed to analyze the potential of anti-PD-L1 antibodies to inhibit the interaction of PD-L1 with its binding partners, PD-1 and CD80. The PD-L1/PD-1 blocking ELISA is considered as the most relevant ELISA by depicting the blocking situation between PD-1 and PD-L1. F_{c}-tagged human PD-L1 (tebu-bio/BPS bioscience) was coated on Maxisorp 96 well plates. After washing and blocking, a fixed concentration of biotinylated human PD-1 (tebu-bio/BPS bioscience) in presence of serial dilutions of anti-PD-L1 hIgG1 or bispecific anti-PD-L1/TA-MUC1 hIgG1 were added thereby competing for the binding to PD-1. After washing, binding of PD-1 was detected by Streptavidin-POD and TMB. As result, the higher the inhibition of the interaction between PD-1 and PD-L1 by anti-PD-L1 antibodies the lower is the resulting OD at 450 nm.

First, a fucose-reduced anti-PD-L1 hIgG1 (PDL-GEX Fuc−) and a fucose-reduced bispecific anti-PD-L1/TA-MUC1 hIgG1 (PM-PDL-GEX Fuc−) were compared to their normal-fucosylated counterparts (PDL-GEX H9D8 and PM-PDL-GEX H9D8) in the PD-L1/PD-1 blocking ELISA (FIG. 2A). Concentration-dependent blocking of PD-1 binding was detected for all four variants tested.

Further, a related blocking ELISA was developed as described above, but instead of PD-1 CD80 ligand, another functionally relevant ligand of PD-L1 was used (FIG. 2B).

### Example 3: A Fucose-Reduced and a Normal-Fucosylated Bispecific Anti-PD-L1/TA-MUC1 hIgG1 Show Comparable Binding to TA-MUC1

The fucose-reduced and the normal-fucosylated bispecific anti-PD-L1/TA-MUC1 hIgG1 showed comparable binding to TA-MUC1. As expected, the monospecific anti-PD-L1 (PDL-GEX H9D8) showed no binding to the cell line ZR-75-1 (FIG. 3).

The binding properties of fucose-reduced and normal-fucosylated bispecific anti-PD-L1/TA-MUC1 hIgG1 (PM-PDL-GEX H9D8 and Fuc−) to human TA-MUC1 expressing tumor cells were analyzed by flow cytometry. The breast cancer cell line ZR-75-1 with strong TA-MUC1 expression, but only minimal or absent PD-L1 expression was used to determine TA-MUC1 binding. Briefly, target cells were harvested and incubated with indicated antibodies in serial dilutions. Afterwards, cells were washed and incubated with a secondary goat anti-hIgG AF488-conjugated antibody at 4° C. in the dark. Cells were analyzed via flow cytometry.

### Example 4: The Fucose-Reduced Variants of an Anti-PD-L1 hIgG1 and a Bispecific Anti-PD-L1/TA-MUC1 hIgG1 Show Increased Binding to FcyRIIIa Compared to the Normal-Fucosylated Variants

The fucose-reduced anti-PD-L1 (PDL-GEX Fuc−) has a decreased EC50 value compared to the normal-fucosylated anti-PD-L1 hIgG1 (PDL-GEX H9D8) demonstrating ˜5-fold enhanced binding to FcγRIIIa of the fucose-reduced variant compared to the normal-fucosylated variant. In contrast, the relative potency of the bispecific fucose-reduced anti-PD-L1/TA-MUC-1 hIgG1 (PM-PDL-GEX Fuc−) was determined as 10.4. From that, also for the bispecific anti-PD-L1/TA-MUC1 hIgG1 binding to FcγRIIIa is enhanced by ˜5-fold for the fucose-reduced variant compared to the normal-fucosylated counterpart (FIG. 4).

Induction of antibody-dependent cell cytotoxicity (ADCC) is connected with antibody binding to the tumor antigen on one site and the recruitment of effector cells via binding of its F_{c} part to FcγIIIa receptors on these cells on the other site. De-fucosylation of hIgG1 is expected to result in higher affinity to FcγRIIIa thereby resulting in stronger ADCC mediated by human peripheral blood mononuclear cells against tumor cells expressing the respective antigen.

In order to characterize binding of the antibody F_{c} part to FcγRIIIa on a molecular level, a new assay using a bead-based technology of Perkin Elmer (AlphaScreen®) was developed. The extracellular domain of recombinant human FcγRIIIa (produced recombinantly by Glycotope in the GEX-H9D8 cell line) was used in this assay. His-tagged FcγRIIIa was captured by Ni-chelate donor beads. The test antibodies and rabbit-anti-mouse coupled acceptor beads compete for binding to FcγRIIIa. In case of interaction of FcγRIIIa with rabbit-anti-mouse acceptor beads only, donor and acceptor beads come into close proximity, which leads upon laser excitation to light emission by chemiluminescence. A maximum signal is achieved. In case of competition of the test antibody binding to FcγRIIIa with the acceptor beads the maximum signal is reduced in a concentration dependent manner. The chemiluminescence was quantified by measurement at 520-620 nm. As a result, a concentration dependent sigmoidal dose-response curve was received, which is defined by top-plateau, bottom-plateau, slope and EC50. The EC50 equals the effective antibody concentration needed for 50% of maximum binding to FcγRIIIa.

### Example 5: A Fucose-Reduced Anti-PD-L1 hIgG1 and a Fucose-Reduced Bispecific Anti-PD-L1/TA-MUC1 hIgG1 Show Increased Killing of TA-MUC+ and PD-L1+ Tumor Cells Compared to their Normal-Fucosylated Counterparts

The fucose-reduced bispecific anti-PD-L1/TA-MUC1 hIgG1 (PM-PDL-GEX Fuc−) showed strongly enhanced ADCC activity compared to the normal-fucosylated bispecific anti-PD-L1/TA-MUC1 hIgG1 against the breast cancer cell line ZR-75-1 which expresses high levels of TA-MUC1 and only marginal levels of PD-L1. The fucose-reduced anti-PD-L1 (PDL-GEX Fuc−) and the fucose-reduced bispecific anti-PD-L1/TA-MUC1 hIgG1 (PM-PDL-GEX Fuc−) mediated strongly enhanced ADCC against PD-L1 positive tumor cells such as the prostate carcinoma cell line DU-145 compared to their normal-fucosylated counterparts.

The capacity to mediate ADCC against tumor cells was analyzed using a europium release assay. Briefly, target cells were loaded with europium (Eu2+) by electroporation and incubated with an FcyRIIIa-transfected NK cell line in the presence of test antibodies for 5 hours with an E:T-ratio of 30:1. Europium release to the supernatant (indicating antibody mediated cell death) was quantified using a fluorescence plate reader. Maximal release was achieved by incubation of target cells with triton-X-100 and spontaneous release was measured in samples containing only target cells but no antibody and no effector cells. Specific cytotoxicity was calculated as: % specific lysis=(experimental release−spontaneous release)/(maximal release−spontaneous release)×100.

First of all, ADCC was analyzed against the breast cancer cell line ZR-75-1 which expresses high levels of TA-MUC1 and only marginal levels of PD-L1 (FIG. 5A, see Example 3).

Second, ADCC was analyzed against the prostate carcinoma cell line DU-145 which strongly expresses PD-L1 and has moderate TA-MUC1 expression (FIGS. 5B and C). PD-L1 and TA-MUC1 expression was analyzed by flow cytometry using PDL-GEX H9D8 and a TA-MUC1-specific antibody, respectively, detected by a fluorochrome-labeled secondary antibody.

Third, ADCC was analyzed again against the prostate carcinoma cell line DU-145 by using fucose-reduced anti-PD-L1 and fucose-reduced bi-specific anti-PD-L1/TA-MUC1 hIgG1 compared to their normal-fucosylated counterparts (FIG. 5D).

### Example 6: A Fucose-Reduced Anti-PD-L1 hIgG1 and a Fucose-Reduced Bispecific Anti-PD-L1/TA-MUC1 hIgG1 Show No ADCC Effect Against PD-L1+ PBMCs

No ADCC effect mediated by fucose-reduced anti-PD-L1 and fucose-reduced bispecific anti-PD-L1/TA-MUC1 against B cells (FIG. 6A) and monocytes was detected (FIG. 6B).

PD-L1 is reported to be expressed not exclusively on tumor cells but also on different immune cells, e.g. monocytes or B cells. Since fucose-reduced anti-PD-L1 and fucose-reduced bispecific anti-PD-L1/TA-MUC1 show strongly increased ADCC effects against tumor cells compared to their normal-fucosylated counterparts, it could be expected that they also mediate ADCC against PD-L1+ immune cells.

Monocytes and B cells are described to express PD-L1, therefore both immune cell populations were analyzed in a FACS based ADCC assays as potential target cells. Briefly, B cells and monocytes were isolated from PBMCs by negative selection via Magnetic-Activated Cell Sorting (MACS) to a purity of >95%. A commercial anti-CD20 mAb (Gazyvaro®, Roche) was used as positive control on B cells as well as on the human Burkitt lymphoma cell line Daudi. For monocytes, staurosporine served as positive control on isolated monocytes as well as the human leukemia monocytic cell line THP-1. B cells, monocytes or positive control cell lines were labelled with Calcein-AM for 20 min at 37° C. followed by washing. Afterwards, cells were seeded in a 96-well round bottom plate and fucose-reduced anti-PD-L1 hIgG1 or fucose-reduced bispecific anti-PD-L1/TA-MUC1 was added at different concentrations. An FcγRIIIa-transfected NK cell line was used as effector cells. After a total incubation time of 4 h at 37° C., cells were stained with 7-AAD and analyzed by flow cytometry.

### Example 7: A Fucose-Reduced and a Normal-Fucosylated Bispecific Anti-PD-L1/TA-MUC1 hIgG1 Show Comparable Results in a Cell Based PD-1/PD-L1 Blockade Bioassay

Comparable dose-dependent release of the PD-1/PD-L1 break was detected for both, the de-(PM-PDL-GEX Fuc−) and normal-fucosylated (PM-PDL-GEX H9D8) bispecific anti-PD-L1/TA-MUC1 hIgG1 in accordance with the PD-L1/PD-1 block ELISA (see example 1). As expected, Nivolumab was effective as positive control (FIG. 7).

The PD-1/PD-L1 blockade bioassay (Promega) is a bioluminescent cell-based assay that can be used to measure the potency of antibodies designed to block the PD-1/PD-L1 interaction. The assay consists of two genetically engineered cell lines:

i. PD-1 positive responder cells with luciferase reporter gene (Jurkat T cells)
ii. PD-L1 positive stimulator CHO-K1 cells

Due to PD-1/PD-L1 interaction the TCR signaling and the resulting NFAT-mediated luciferase activity in the responder cells is inhibited. This inhibition can be reversed in presence of antibodies blocking either the PD-1 or PD-L1 producing a luminescent signal which can be detected in a luminescent reader.

### Example 8: A Fucose-Reduced and a Normal-Fucosylated Bispecific Anti-PD-L1/TA-MUC1 hIgG1 and a Fucose-Reduced Anti-PD-L1 hIgG1 Induces Comparable IL-2 in a Allogeneic Mixed Lymphocyte Reaction (MLR)

No influence of de-fucosylation on IL-2 secretion was detected since the fucose-reduced (PM-PDL-GEX Fuc−) and the normal-fucosylated bispecific anti-PD-L1/TA-MUC1 hIgG1 (PM-PDL-GEX H9D8) and the fucose-reduced anti-PD-L1 hIgG1 (PDL-GEX Fuc−) induced comparable amount of IL-2.

The mixed lymphocyte reaction (MLR) is a functional assay which was established to analyze the effect of PD-L1 blocking antibodies on the suppression of PD-1 expressing T cells by PD-L1 expressing antigen presenting cells. The assay measures the response of T cells (either isolated T cells or PBMCs) from one donor as responders to monocyte-derived dendritic cells (moDCs) from another donor as stimulators (=allogenic MLR).

Briefly, monocytes were isolated from buffy coat via negative selection using magnetic-activated cell sorting and then differentiated to moDCs with IL-4 and GM-CSF for 7 days. Then, the phenotype of moDCs was analyzed by flow cytometry (FIG. 8A).

Additionally, after differentiation, moDCs were cultivated with isolated T cells with a stimulator/responder-ratio of 1:10. After 3 days, supernatants were harvested for an IL-2 ELISA (Affimetryx eBioscience) (FIG. 8B).

### Example 9: A Fucose-Reduced Anti-PD-L1 hIgG1 and Fucose-Reduced Bispecific Anti-PD-L1/TA-MUC1 hIgG1 Shows Increased T Cell Activation Compared to Normal-Fucosylated Counterparts and an Anti-PD-L1 Antibody with No/Weak FcyR-Binding Capacity

A fucose-reduced anti-PD-L1 hIgG1 (PDL-GEX Fuc−) and a fucose-reduced bispecific anti-PD-L1/TA-MUC1 hIgG1 (PM-PDL-GEX Fuc−) induces enhanced T cell activation compared to normal-fucosylated anti-PD-L1 hIgG1 (PDL-GEX H9D8) and bispecific anti-PD-L1/TA-MUC1 hIgG1 (PM-PDL-GEX H9D8), and compared to an anti-PD-L1 antibody with no/weak FcyR-binding capacity (Atezolizumab) in an allogeneic MLR.

CD8 T cells (CD3⁺CD8⁺ cells) of allogeneic MLRs with moDCs and isolated T cells from three different donors (FIGS. 9A, B and C) in presence of 1 μg/ml test antibody were analyzed on day 5 for activation via expression of CD25 by flow cytometry. A MLR without addition of antibody served as negative control.

The fact that fucose-reduced anti-PD-L1 and anti-PD-L1/TA-MUC1 antibodies induced increased T cell activation is surprising, since no differences between the glycosylation variants were seen in the blocking ELISA (see Example 2), in the PD-1/PD-L1 blockade bioassay (see Example 7) and in the IL-2 secretion (see Example 8). Increased activation of T cells due to fucose-reduced anti-PD-L1 hIgG1 and fucose-reduced bispecific anti-PD-L1/TA-MUC1 hIgG1 is observed with T cells of different donors and is expected to be a general effect.

The finding that fucose-reduced monospecific anti-PD-L1 (PDL-GEX Fuc−) and bispecific anti-PD-L1/TA-MUC1 hIgG1 (PM-PDL-GEX Fuc−) induces enhanced CD8 T cell activation is important, since CD8 T cells represent cytotoxic T cells which play a crucial role in the anti-tumor response and have the capacity to directly kill cancer cells.

### Example 10: A Fucose-Reduced Anti-PD-L1 hIgG1 and Fucose-Reduced Bispecific Anti-PD-L1/TA-MUC1 hIgG1 Shows Increased T Cell Activation Compared to Normal-Fucosylated Counterparts and an Anti-PD-L1 with No/Weak FcyR-Binding Capacity in a MLR with Isolated T Cells and Total PBMCs

The fucose-reduced anti-PD-L1 hIgG1 (PDL-GEX Fuc−) and fucose-reduced bispecific anti-PD-L1/TA-MUC1 hIgG1 (PM-PDL-GEX Fuc−) induced stronger CD8 T cell activation compared to normal-fucosylated anti-PD-L1 hIgG1 (PDL-GEX H9D8), to a bispecific anti-PD-L1/TA-MUC1 hIgG1 (PM-PDL-GEX H9D8) and compared to an anti-PD-L1 with no/weak FcyR-binding capacity (Atezolizumab) measured by expression of CD25 and CD137 on CD3⁺CD8⁺ cells using either T cells or PBMCs as responder cells in the MLR.

Further, cultivation of moDCs with PBMCs additionally leads to increased CD4 T cell activation (CD3⁺CD8⁻ cells ergo CD4 T cells) measured by expression of CD25 and CD137, which was not observed earlier in MLRs using isolated T cells. The usage of PBMCs, which contain NK cells, instead of isolated T cells shows that NK cells or a potential NK cell-mediated ADCC effect on PD-L1+ cells has no negative impact on T cell activation.

In an allogeneic MLR, isolated T cells or PBMCs were cultivated for 5 d with moDCs in presence of 1 μg/ml test antibody. A MLR without addition of antibody served as negative control. Then, CD8 T cell activation was measured by the expression of CD25 and CD137 on CD8 T cells for the MLR with isolated T cells (FIGS. 10A and B) and for the MLR with PBMCs (FIGS. 10C and D). CD4 T cell activation was also measured by the expression of CD25 and CD137 on CD4 T cells for the MLR with PBMCs (FIGS. 10E and F).

### Example 11: A Fucose-Reduced Anti-PD-L1 hIgG1 and Fucose-Reduced Bispecific Anti-PD-L1/TA-MUC1 hIgG1 Also Increases CD69 Expression on T Cells

The fucose-reduced anti-PD-L1 hIgG1 (PDL-GEX Fuc−) and fucose-reduced bispecific anti-PD-L1/TA-MUC1 hIgG1 (PM-PDL-GEX Fuc−) induce stronger CD69 expression on CD8 T cells compared to normal-fucosylated anti-PD-L1 hIgG1 (PDL-GEX H9D8) and bispecific anti-PD-L1/TA-MUC1 hIgG1 (PM-PDL-GEX H9D8) (FIG. 11).

D8 T cells (CD3⁺CD8⁺ cells) of an allogeneic MLR with isolated T cells and moDCs in presence of 1 μg/ml test antibody were analyzed for CD69 expression on day 5 via flow cytometry. A MLR without addition of antibody served as negative control. CD69 is an additional activation marker beside CD25 and CD137.

### Example 12: FcyRs Play a Crucial Role for the Activation of T Cells Via Blockade of PD-L1

This allogeneic MLR shows that FcyR-binding plays a crucial role for the increased activation of T cells using a fucose-reduced anti-PD-L1 antibody. The increased T cell activation due to a fucose-reduced anti-PD-L1 hIgG1 (PDL-GEX Fuc−) was inhibited to a level comparable to the normal-fucosylated anti-PD-L1 hIgG1 (PDL-GEX H9D8) or non-glycosylated anti-PD-L1 hIgG1 with no/weak FcyR-binding capacity (Atezolizumab) due to addition of another fucose-reduced antibody with an irrelevant specificity (termed as block) (the antigen is not present in the MLR) (FIG. 12).

In this allogeneic MLR with moDCs and isolated T cells, the fucose-reduced antibody with irrelevant specificity (termed as block) was added in ten times higher concentration compared to fucose-reduced anti-PD-L1 hIgG1 and therefore blocks the binding of fucose-reduced anti-PD-L1 hIgG1 to the FcyRs. This experiment demonstrates the important role of FcγRs for the increased T cell activation due to fucose-reduced anti-PD-L1 antibodies.

### Example 13: In Presence of a De-Fucosylated Anti-PD-L1 hIgG1 Dendritic Cells Show a More Mature Phenotype Compared to a Normal-Fucosylated Anti-PD-L1 hIgG1

In presence of a fucose-reduced anti-PD-L1 hIgG1 (PDL-GEX Fuc−), moDCs showed less expression of CD14 compared to a normal-fucosylated anti-PD-L1 hIgG1 (PDL-GEX H9D8). In contrast, CD16 (FcγRIII) and the co-stimulatory molecules CD40 and CD86, and the DC-marker CD83 were expressed in higher levels in presence of a fucose-reduced anti-PD-L1 hIgG1 compared to a normal-fucosylated anti-PD-L1 hIgG1.

MoDCs of this MLR were analyzed on day 5 for the surface expression of different marker such as CD14 (FIG. 13A), CD16 (FIG. 13B), CD40 (FIG. 13C), CD86 (FIG. 13E) and CD83 (FIG. 13D) using flow cytometry.

This example shows that fucose-reduced anti-PD-L1 hIgG1 antibodies have a positive effect on the maturation status of DCs.

### Example 14: T Cell Activation Measured by Cytotoxicity of a Normal-Fucosylated Anti-PDL1 hIgG1 and a Fucose-Reduced Anti-PDL1 hIgG1

In order to analyze whether increased T cell activation due to a fucose-reduced anti-PD-L1 results in a benefit in functionality, T cells which were activated in a allogeneic MLR from the same different donors as indicated in Example 9 in absence or presence of PDL-GEX H9D8, PDL-GEX Fuc− and Atezolizumab [1 μg/ml] were harvested and afterwards their cytotoxic capacity was determined using a europium release assay. Briefly, the cancer cell line ZR-75-1 as target cells were loaded with europium (Eu2+) by electroporation and incubated with harvested T cells for 5 hours with an E:T-ratio of 50:1 (E:T-ratio=effector:target-ratio, effector=T cells; target=ZR-75-1). Europium release to the supernatant (indicating lysis of target cells) was quantified using a fluorescence plate reader. Cytotoxicity is indicated as fold change compared to unstimulated T cells (T cells without stimulation due to allogeneic moDCs).

Activation of T cells with PDL-GEX Fuc− resulted in increased cytotoxicity compared to PDL-GEX H9D8, Atezolizumab and medium control (medium control=T cells after a MLR without addition of test antibody) (FIG. 14).

### Example 15: Detection of T Cell Activation by Using Fucose-Reduced Anti-PD-L1 hIgG1 (PDL-GEX Fuc−) Having Different Amounts of Core-Fucosylation

To figure out the most promising amount of core-fucosylation for PDL-GEX Fuc−, PDL-GEX H9D8 having 89% core-fucosylated N-glycans are mixed with PDL-GEX having 4% core-fucosylated N-glycans to simulate different amounts of core-fucosylation. The antibodies or rather the antibody mixture were/was tested for T cell activation in a MLR-assay with isolated T cells of one donor as responders to monocyte-derived dendritic cells (moDCs) from another donor as stimulators. Read-out was the CD25- and CD137 expression on CD8 T cells (FIG. 15).

### Example 16: Comparable Antigen Binding of Anti-PD-L1 Antibodies with Mutations in their F_{c} Part to their Non-Mutated Counterpart

Two normal-fucosylated anti-PD-L1 antibodies were generated with mutations in their F_{c} parts. First, an anti-PD-L1 antibody with three amino acid changes: S239D, 1332E and G236A according to EU nomenclature (termed PDL-GEX H9D8 mut1). Second, an anti-PD-L1 antibody with five amino acid changes: L235V, F243L, R292P, Y300L and P396L according to EU nomenclature (termed PDL-GEX H9D8 mut2).

PDL-GEX H9D8 mut1 and PDL-GEX H9D8 mut2 were tested for their binding to PD-L1 in comparison to the non-mutated PDL-GEX H8D8 in an antigen ELISA. Therefore, human PD-L1 was coated on Maxisorp 96 well plates. After washing and blocking, serial dilutions of test antibodies were added. After washing, binding of test antibody was determined using POD-coupled secondary antibody and TMB.

No obvious difference in PD-L1 binding was observed between PDL-GEX H9D8, PDL-GEX H9D8 mut1 and PDL-GEX H9D8 mut2 (FIG. 16).

### Example 17: Increased FcyRIIIa Engagement of Anti-PD-L1 Antibodies with Mutations in their F_{c} Part Compared to their Non-Mutated Counterpart

Binding of antibody F_{c} part to FcyRIIIa was analyzed using a bead-based technology of Perkin Elmer (AlphaScreen®) as described in Example 4. In case of interaction of FcγRIIIa with the F_{c} part of the test antibody, the signal is reduced in a concentration dependent manner.

PM-PDL-GEX H9D8 mut1 and PM-PDL-GEX H9D8 mut2 showed increased binding to FcyRIIIa compared to the non-mutated PDL-GEX H9D8 visualized by the shift to lower effective concentrations (FIG. 17).

### Example 18: Increased T Cell Activation of Anti-PD-L1 Antibodies with Mutations in their F_{c} Part Compared to their Non-Mutated Counterpart

T cell activation of the normal-fucosylated F_{c}-mutated PDL-GEX H9D8 mut1 and PDL-GEX H9D8 mut2 was determined in an allogeneic MLR as described in Example 9 in comparison to the normal-fucosylated non-mutated PDL-GEX H9D8 and to the de-fucosylated non-mutated PDL-GEX Fuc−.

PM-PDL-GEX mut1 and PDL-GEX mut2 showed increased T cell activation in comparison to PDL-GEX H9D8 demonstrating that enhanced T cell activation can be achieved by using either a de-fucosylated anti-PD-L1 antibody (PDL-GEX Fuc−) or by using anti-PD-L1 antibodies comprising sequence mutations leading to enhanced binding FcyRIIIa (FIG. 18).

### Example 19: Enhanced T Cell Activation Due to a De-Fucoslyated Anti-PD-L1 Antibody is Also Visualized by Proliferation

The proliferation of CD8 T cells in a MLR was determined on day 5 by carboxyfluorescein succinimidyl ester (CFSE) dilution measured by flow cytometric analysis. Therefore, cells were labeled with CFSE. Proliferating cells show a decreased CFSE-signal due to cell division.

The de-fucosylated anti-PD-L1 antibody (PDL-GEX Fuc−) showed increased proliferation of CD8 T cells compared to normal-fucosylated anti-PD-L1 antibody (PDL-GEX H9D8) and compared to a non-glycosylated anti-PD-L1 (Atezolizumab) (FIG. 19).

### Example 20: Enhanced T Cell Activation Due to a De-Fucoslyated Anti-PD-L1 Antibody and a De-Fucosylated Bispecific Anti-PD-L1/TA-MUC1 Antibody Observed in Presence of Cancer Cells

A de-fucosylated anti-PD-L1 (PDL-GEX Fuc−) and de-fucosylated bispecific anti-PD-L1/TA-MUC1 antibody (PM-PDL-GEX Fuc−) were compared for their ability to induce T cell activation in presence of cancer cells in a MLR. Therefore, various cancer cells lines were added in the MLR (T cells:moDC:cancer cell-ratio=100:10:1).

Measuring CD25 expression on CD8 T cells revealed that the presence of HSC-4 and ZR-75-1 had no obvious effect on the CD8 T cell activation, whereas Ramos cells appear to have some suppressive impact. However, the augmented activation by PDL-GEX Fuc− and PM-PDL-GEX Fuc− were observed in presence of all cancer cell lines tested (FIG. 20).

### Example 21: PDL-GEX CDR Mutants Show Comparable Binding and Blocking Capacity Compared to the Non-Mutated Counterpart

Different CDR mutants of PDL-GEX Fuc− were generated:

### PDL-GEX Fuc− CDRmut a (SEQ ID NO. 60+SEQ ID NO. 68)

### PDL-GEX Fuc− CDRmut b (SEQ ID NO. 62+SEQ ID NO. 69)

### PDL-GEX Fuc− CDRmut c (SEQ ID NO. 63+SEQ ID NO. 70)

### PDL-GEX Fuc− CDRmut d (SEQ ID NO. 64)

### PDL-GEX Fuc− CDRmut e (SEQ ID NO. 65+SEQ ID NO. 71)

### PDL-GEX Fuc− CDRmut f (SEQ ID NO. 66+SEQ ID NO. 72)

### PDL-GEX Fuc− CDRmut g (SEQ ID NO. 63+SEQ ID NO. 72)

### PDL-GEX Fuc− CDRmut h (SEQ ID NO. 67+SEQ ID NO. 74)

### PDL-GEX Fuc− CDRmut i (SEQ ID NO. 63+SEQ ID NO. 68)

and tested I) for their PD-L1 binding capacity using PD-L1 expressing DU-145 and flow cytometric analysis and II) for their blocking capacity in an PD-L1/PD-1 blocking ELISA as descripted in Example 2. All CDR mutants showed comparable binding and blocking compared to the non-mutated PDL-GEX Fuc− (FIGS. 21A and B).

### Example 22: PM-PDL-GEX CDR Mutants Show Comparable Binding and Blocking Capacity Compared to the Non-Mutated Counterpart

Different CDR mutants of PM-PDL-GEX Fuc− were generated:

### PM-PDL-GEX Fuc− CDRmut a (SEQ ID No. 64)

### PM-PDL-GEX Fuc− CDRmut b (SEQ ID NO. 66+SEQ ID NO. 72),

and tested in various assays:
I) For their PD-L1 binding capacity using PD-L1 antigen ELISA. Therefore, human PD-L1 was coated on Maxisorp 96 well plates. After washing and blocking, serial dilutions of test antibodies were added. After washing, binding of test antibody was determined using POD-coupled secondary antibody and TMB (FIG. 22A).
II) For their blocking capacity in an PD-L1/PD-1 blocking ELISA as descripted in Example 2 (FIG. 22B).
III) For their TA-MUC1 binding capacity using TA-MUC1 expressing T-47D and flow cytometric analysis (FIG. 22C).

Mutation of the CDR part had no obvious effect on PM-PDL-GEX binding to PD-L1, blocking of PD-L1/PD1 interaction and TA-MUC1 binding.

### Example 23: PM-PDL-GEX CDR Mutants Show Comparable Enhanced Activation of CD8 T Cells to the Non-Mutated Counterparts

Different CDR mutants of PM-PDL-GEX H9D8 and PM-PDL-GEX Fuc− were generated:

### PM-PDL-GEX H9D8 CDRmut a (SEQ ID No. 64)

### PM-PDL-GEX H9D8 CDRmut b (SEQ ID NO. 66+SEQ ID NO. 72)

### PM-PDL-GEX Fuc− CDRmut a (SEQ ID No. 64)

### PM-PDL-GEX Fuc− CDRmut b (SEQ ID NO. 66+SEQ ID NO. 72),

and tested for their capacity to activate T cells in an allogeneic MLR as described in Example 9. The CDR mutated PM-PDL-GEX Fuc− variants activated CD8 T cells (CD25+ cells of CD8 T cells) comparable to non-mutated PM-PDL-GEX Fuc−. The CDR mutated PM-PDL-GEX H9D8 variants activated CD8 T cells comparable to non-mutated PM-PDL-GEX H9D8 (FIG. 23).

1. An antibody, which effects enhanced T cell activation in comparison to a reference antibody being glycosylated including more than 80% core-fucosylation.

2. The antibody of claim 1, wherein the reference antibody is obtainable from CHOdhfr− (ATCC No. CRL-9096).

3. The antibody of claim 1, which effects enhanced T cell activation in comparison to a reference antibody being non-glycosylated.

4. The antibody of claim 1, wherein T cell activation is effected by an antibody characterized by enhanced binding to FcγRIIIa.

5. The antibody of claim 1, wherein said antibody is glycosylated, but essentially lacks core-fucosylation, and wherein said glycosylation is preferably human glycosylation.

6. (canceled)

7. The antibody of claim 1, wherein said glycosylation of said reference antibody is human glycosylation.

8. The antibody of claim 5, which is from 0% to 80% fucosylated, preferably wherein said antibody is obtainable from the cell line NM-H9D8-E6 (DSM ACC 2807), NM-H9D8-E6Q12 (DSM ACC 2856), or a cell or cell line derived therefrom.

9. (canceled)

10. The antibody of claim 1, wherein said antibody comprises one or more sequence mutations, wherein the binding of said antibody to FcγRIIIa is increased compared to a non-mutated antibody, preferably wherein said antibody comprises one or more sequence mutations selected from S238D, S239D, I332E, A330L, S298A, E333A, L334A, G236A and L235V according to EU-nomenclature.

11. (canceled)

12. The antibody of claim 1, wherein said T cell activation is accompanied by maturation of dendritic cells and/or expression of co-stimulatory molecules and maturation markers.

13. The antibody of claim 1, wherein said T cell activation is detectable by the expression of CD25, CD69 and/or CD137.

14. The antibody of claim 1, wherein said antibody is a PD-L1 antibody, preferably wherein said antibody is a bifunctional monospecific antibody comprising a F_{c} region.

15. (canceled)

16. The antibody of claim 14, wherein said antibody is a trifunctional bispecific antibody comprising a F_{c} region.

17. The antibody of claim 14, wherein said antibody further binds to a cancer antigen, preferably wherein said cancer antigen is TA-MUC1.

18. (canceled)

19. (canceled)

20. The antibody of claim 1, wherein said antibody is a TA-MUC1 antibody, preferably wherein said antibody is a bifunctional monospecific antibody comprising a F_{c} region.

21. (canceled)

22. The antibody of claim 20, wherein said antibody is a trifunctional bispecific antibody comprising a F_{c} region.

23. The antibody of claim 22, wherein said antibody further binds to an immune checkpoint protein, preferably wherein said immune checkpoint protein is PD-L1.

24. (canceled)

25. (canceled)

26. The antibody of claim 23, wherein the antibody comprises single chain Fᵥ regions binding to PD-L1, preferably wherein the single chain Fᵥ regions are coupled to the constant domain of the light chain or to the CH₃ domain of the F_{c} region.

27. The antibody of claim 23, wherein said antibody comprises V_{H} and V_{L} domains binding to TA-MUC1.

28. (canceled)

29. The antibody of claim 1 for use in therapy.

30. The antibody of claim 1 for use in a method for activating T-cells, preferably wherein the activation of T-cells is for the treatment of cancer disease, inflammatory disease, virus infectious disease and autoimmune disease.

31. (canceled)

32. (canceled)

33. (canceled)

34. (canceled)

35. (canceled)


SEQUENCE LISTING (83 sequences)

SEQ ID NO: 1 - protein, 10 aa, Artificial sequence
  CDR H1
GFTFSDSWIH

SEQ ID NO: 2 - protein, 18 aa, Artificial sequence
  CDR H2
AWISPYGGSTYYADSVKG

SEQ ID NO: 3 - protein, 9 aa, Artificial sequence
  CDR H3
RHWPGGFDY

SEQ ID NO: 4 - protein, 11 aa, Artificial sequence
  CDR L1
RASQDVSTAVA

SEQ ID NO: 5 - protein, 7 aa, Artificial sequence
  CDR L2
SASFLYS

SEQ ID NO: 6 - protein, 9 aa, Artificial Sequence
  CDR L3
QQYLYHPAT

SEQ ID NO: 7 - protein, 5 aa, Artificial sequence
  CDR H1
NYWMN

SEQ ID NO: 8 - protein, 19 aa, Artificial sequence
  CDR H2
EIRLKSNNYTTHYAESVKG

SEQ ID NO: 9 - protein, 6 aa, Artificial sequence
  CDR H3
HYYFDY

SEQ ID NO: 10 - protein, 16 aa, Artificial sequence
  CDR L1
RSSKSLLHSNGITYFF

SEQ ID NO: 11 - protein, 7 aa, Artificial sequence
  CDR L2
QMSNLAS

SEQ ID NO: 12 - protein, 9 aa, Artificial sequence
  CDR L3
AQNLELPPT

SEQ ID NO: 13 - protein, 486 aa, Artificial sequence
  Light chain coupled to scFv region
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKSKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 14 - protein, 447 aa, Artificial sequence
  Heavy chain
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 15 - protein, 713 aa, Artificial sequence
  Heavy chain coupled to scFv region
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGKGGGGSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 16 - protein, 220 aa, Artificial sequence
  Light chain
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKSKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 118 aa, Artificial sequence
  VH domain of the PD-L1 binding site
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 18 - protein, 108 aa, Artificial sequence
  VL domain of the PD-L1 binding site
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 19 - protein, 117 aa, Artificial sequence
  VH domain of TA-MUC1 binding site
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 20 - protein, 114 aa, Artificial sequence
  VL domain of the TA-MUC1 binding site
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKSK

SEQ ID NO: 21 - protein, 25 aa, Artificial sequence [sequence omitted]

SEQ ID NO: 22 - protein, 13 aa, Artificial sequence
  Frame work region 2 VH domain of the PD-L1 binding site
WVRQAPGKGLEWV

SEQ ID NO: 23 - protein, 32 aa, Artificial sequence
  Frame work region 3 VH domain of the PD-L1 binding site
RFTISADTSKNTAYLQMNSLRAEDTAVYYCAR

SEQ ID NO: 24 - protein, 11 aa, Artificial sequence
  Frame work region 4 VH domain of the PD-L1 binding site
WGQGTLVTVSA

SEQ ID NO: 25 - protein, 23 aa, Artificial sequence
  Frame work region 1 VL domain of the PD-L1 binding site
DIQMTQSPSSLSASVGDRVTITC

SEQ ID NO: 26 - protein, 15 aa, Artificial sequence
  Frame work region 2 VL domain of the PD-L1 binding site
WYQQKPGKAPKLLIY

SEQ ID NO: 27 - protein, 32 aa, Artificial sequence
  Frame work region 3 VL domain of the PD-L1 binding site
GVPSRFSGSGSGTDFTLTISSLQPEDFATYYC

SEQ ID NO: 28 - protein, 11 aa, Artificial sequence
  Frame work region 4 VL domain of the PD-L1 binding site
FGQGTKVEIKR

SEQ ID NO: 29 - protein, 30 aa, Artificial sequence
  Frame work region 1 VH domain of the TA-MUC1 binding site
EVQLVESGGGLVQPGGSMRLSCVASGFPFS

SEQ ID NO: 30 - protein, 14 aa, Artificial sequence
  Frame work region 2 VH domain of the TA-MUC1 binding site
WVRQAPGKGLEWVG

SEQ ID NO: 31 - protein, 32 aa, Artificial sequence
  Frame work region 3 VH domain of the TA-MUC1 binding site
RFTISRDDSKNSLYLQMNSLKTEDTAVYYCTR

SEQ ID NO: 32 - protein, 11 aa, Artificial sequence
  Frame work region 4 VH domain of the TA-MUC1 binding site
WGQGTLVTVSS

SEQ ID NO: 33 - protein, 23 aa, Artificial sequence
  Frame work region 1 VL domain of the TA-MUC1 binding site
DIVMTQSPLSNPVTPGEPASISC

SEQ ID NO: 34 - protein, 15 aa, Artificial sequence
  Frame work region 2 VL domain of the TA-MUC1 binding site
WYLQKPGQSPQLLIY

SEQ ID NO: 35 - protein, 32 aa, Artificial sequence
  Frame work region 3 VL domain of the TA-MUC1 binding site
GVPDRFSGSGSGTDFTLRISRVEAEDVGVYYC

SEQ ID NO: 36 - protein, 12 aa, Artificial sequence
  Frame work region 4 VL domain of the TA-MUC1 binding site
FGQGTKVEIKSK

SEQ ID NO: 37 - protein, 485 aa, Artificial sequence
  Light chain coupled to scFv region
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 38 - protein, 219 aa, Artificial sequence
  Light chain
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 113 aa, Artificial sequence
  VL domain of TA-MUC1 binding site
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKR

SEQ ID NO: 40 - protein, 448 aa, Artificial sequence
  Heavy chain PDL-GEX
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 41 - protein, 448 aa, Artificial sequence
  Heavy chain PDL-GEX variant
EVQLVESGGGLVQPGGSLRLSCAASGFTISDSWIHWVRQAPGKGLEWVAWITPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 42 - protein, 448 aa, Artificial sequence
  Heavy chain PDL-GEX variant
EVQLVESGGGLVQPGGSLRLSCAASAFTFSDSWIHWVRQAPGKGLEWVGWISPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 43 - protein, 448 aa, Artificial sequence
  Heavy chain PDL-GEX variant
EVQLVESGGGLVQPGGSLRLSCAASGFTFSDSWMHWVRQAPGKGLEWVAWLSPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 44 - protein, 448 aa, Artificial sequence
  Heavy chain PDL-GEX variant
EVQLVESGGGLVQPGGSLRLSCAASAFTFSESWIHWVRQAPGKGLEWVAWISPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 45 - protein, 448 aa, Artificial sequence
  Heavy chain PDL-GEX variant
EVQLVESGGGLVQPGGSLRLSCAASGFTFSESWIHWVRQAPGKGLEWVAWISPYGGSTYYADSLKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 46 - protein, 448 aa, Artificial sequence
  Heavy chain PDL-GEX_variant
EVQLVESGGGLVQPGGSLRLSCAASGFSFSDSWIHWVRQAPGKGLEWVAWISPYGGSTYYADTVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 47 - protein, 448 aa, Artificial sequence
  Heavy chain PDL-GEX_variant
EVQLVESGGGLVQPGGSLRLSCAASGFTFSDSWMHWVRQAPGKGLEWVAWISPYGGSTYYADTVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 48 - protein, 448 aa, Artificial sequence
  Heavy chain PDL-GEX_variant
EVQLVESGGGLVQPGGSLRLSCAASGFTFSDTWIHWVRQAPGKGLEWVAWISPYGGTTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 49 - protein, 448 aa, Artificial sequence
  Heavy chain PDL-GEX_variant
EVQLVESGGGLVQPGGSLRLSCAASGFTFSDSWMHWVRQAPGKGLEWVAWITPYGGSTYYADSVKGRFTISADTSKNTAYLQMNSLRAEDTAVYYCARRHWPGGFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 50 - protein, 214 aa, Artificial sequence
  Light chain PDL-GEX
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 118 aa, Artificial sequence
  VH domain of the PD-L1 binding site_variant
EVQLVESGGGLVQPGGSLRLSCAASGFTISDSWIHWVRQAPGKGLEWVAWITPYGGSTYYADSVKGRFTISADTSKNTAYLQMNSLRAEDTAVYYCARRHWPGGFDYWGQGTLVTVSA

SEQ ID NO: 52 - protein, 118 aa, Artificial sequence
  VH domain of the PD-L1 binding site_variant
EVQLVESGGGLVQPGGSLRLSCAASAFTFSDSWIHWVRQAPGKGLEWVGWISPYGGSTYYADSVKGRFTISADTSKNTAYLQMNSLRAEDTAVYYCARRHWPGGFDYWGQGTLVTVSA

SEQ ID NO: 53 - protein, 118 aa, Artificial sequence
  VH domain of the PD-L1 binding site_variant
EVQLVESGGGLVQPGGSLRLSCAASGFTFSDSWMHWVRQAPGKGLEWVAWLSPYGGSTYYADSVKGRFTISADTSKNTAYLQMNSLRAEDTAVYYCARRHWPGGFDYWGQGTLVTVSA

SEQ ID NO: 54 - protein, 118 aa, Artificial sequence
  VH domain of the PD-L1 binding site_variant
EVQLVESGGGLVQPGGSLRLSCAASAFTFSESWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 55 - protein, 118 aa, Artificial sequence
  VH domain of the PD-L1 binding site_variant
EVQLVESGGGLVQPGGSLRLSCAASGFTFSESWIHWVRQAPGKGLEWVAWISPYGGSTYYADSLKGRFTISADTSKNTAYLQMNSLRAEDTAVYYCARRHWPGGFDYWGQGTLVTVSA

SEQ ID NO: 56 - protein, 118 aa, Artificial sequence
  VH domain of the PD-L1 binding site_variant
EVQLVESGGGLVQPGGSLRLSCAASGFSFSDSWIHWVRQAPGKGLEWVAWISPYGGSTYYADTVKGRFTISADTSKNTAYLQMNSLRAEDTAVYYCARRHWPGGFDYWGQGTLVTVSA

SEQ ID NO: 57 - protein, 118 aa, Artificial sequence
  VH domain of the PD-L1 binding site_variant
EVQLVESGGGLVQPGGSLRLSCAASGFTFSDSWMHWVRQAPGKGLEWVAWISPYGGSTYYADTVKGRFTISADTSKNTAYLQMNSLRAEDTAVYYCARRHWPGGFDYWGQGTLVTVSA

SEQ ID NO: 58 - protein, 118 aa, Artificial sequence
  VH domain of the PD-L1 binding site_variant
EVQLVESGGGLVQPGGSLRLSCAASGFTFSDTWIHWVRQAPGKGLEWVAWISPYGGTTYYADSVKGRFTISADTSKNTAYLQMNSLRAEDTAVYYCARRHWPGGFDYWGQGTLVTVSA

SEQ ID NO: 59 - protein, 118 aa, Artificial sequence
  VH domain of the PD-L1 binding site_variant
EVQLVESGGGLVQPGGSLRLSCAASGFTFSDSWMHWVRQAPGKGLEWVAWITPYGGSTYYADSVKGRFTISADTSKNTAYLQMNSLRAEDTAVYYCARRHWPGGFDYWGQGTLVTVSA

SEQ ID NO: 60 - protein, 10 aa, Artificial sequence
  CDR1 in the VH domain of the PD-L1 binding site _variant
GFTISDSWIH

SEQ ID NO: 61 - protein, 10 aa, Artificial sequence
  CDR1 in the VH domain of the PD-L1 binding site_variant
GFTISSSWIH

SEQ ID NO: 62 - protein, 10 aa, Artificial sequence
  CDR1 in the VH domain of the PD-L1 binding site_variant
AFTFSDSWIH

SEQ ID NO: 63 - protein, 10 aa, Artificial sequence
  CDR1 in the VH domain of the PD-L1 binding site_varinat
GFTFSDSWMH

SEQ ID NO: 64 - protein, 10 aa, Artificial sequence
  CDR1 in the VH domain of the PD-L1 binding site_variant
AFTFSESWIH

SEQ ID NO: 65 - protein, 10 aa, Artificial sequence
  CDR1 in the VH domain of the PD-L1 binding site_variant
GFTFSESWIH

SEQ ID NO: 66 - protein, 10 aa, Artificial sequence
  CDR1 in the VH domain of the PD-L1 binding site_variant
GFSFSDSWIH

SEQ ID NO: 67 - protein, 10 aa, Artificial sequence
  CDR1 in the VH domain of the PD-L1 binding site_variant
GFTFSDTWIH

SEQ ID NO: 68 - protein, 18 aa, Artificial sequence
  CDR2 in the VH domain of the PD-L1 binding site_variant
AWITPYGGSTYYADSVKG

SEQ ID NO: 69 - protein, 18 aa, Artificial sequence
  CDR2 in the VH domain of the PD-L1 binding site_variant
GWISPYGGSTYYADSVKG

SEQ ID NO: 70 - protein, 18 aa, Artificial sequence
  CDR2 in the VH domain of the PD-L1 binding site_variant
AWLSPYGGSTYYADSVKG

SEQ ID NO: 71 - protein, 18 aa, Artificial sequence
  CDR2 in the VH domain of the PD-L1 binding site_variant
AWISPYGGSTYYADSLKG

SEQ ID NO: 72 - protein, 18 aa, Artificial sequence
  CDR2 in the VH domain of the PD-L1 binding site_variant
AWISPYGGSTYYADTVKG

SEQ ID NO: 73 - protein, 18 aa, Artificial sequence
  CDR2 in the VH domain of the PD-L1 binding site_variant
AWISPYGGSTYYADSVKA

SEQ ID NO: 74 - protein, 18 aa, Artificial sequence
  CDR2 in the VH domain of the PD-L1 binding site_variant
AWISPYGGTTYYADSVKG

SEQ ID NO: 75 - protein, 18 aa, Artificial sequence
  CDR2 in the VH domain of the PD-L1 binding site_variant
AWISPSGGSTYYADSVKG

SEQ ID NO: 76 - protein, 485 aa, Artificial sequence
  Light chain coupled to scFv region_variant
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGFTISDSWIHWVRQAPGKGLEWVAWITPYGGSTYYADSVKGRFTISADTSKNTAYLQMNSLRAEDTAVYYCARRHWPGGFDYWGQGTLVTVSAGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 77 - protein, 485 aa, Artificial sequence
  Light chain coupled to scFv region_variant
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASAFTFSESWIHWVRQAPGKGLEWVAWISPYGGSTYYADSVKGRFTISADTSKNTAYLQMNSLRAEDTAVYYCARRHWPGGFDYWGQGTLVTVSAGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 78 - protein, 485 aa, Artificial sequence
  Light chain coupled to scFv region_variant
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGFSFSDSWIHWVRQAPGKGLEWVAWISPYGGSTYYADTVKGRFTISADTSKNTAYLQMNSLRAEDTAVYYCARRHWPGGFDYWGQGTLVTVSAGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 79 - protein, 485 aa, Artificial sequence
  Light chain coupled to scFv region_variant
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSEVQLVESGGGLVQPGGSLRLSCAASGFTFSDTWIHWVRQAPGKGLEWVAWISPYGGTTYYADSVKGRFTISADTSKNTAYLQMNSLRAEDTAVYYCARRHWPGGFDYWGQGTLVTVSAGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 80 - protein, 713 aa, Artificial sequence
  Heavy chain coupled to scFv region_variant
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGKGGGGSGGGGSGGGGSGGGGSEVQLVESGGGLVQPGGSLRLSCAASGFTISDSWIHWVRQAPGKGLEWVAWITPYGGSTYYADSVKGRFTISADTSKNTAYLQMNSLRAEDTAVYYCARRHWPGGFDYWGQGTLVTVSAGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 81 - protein, 713 aa, Artificial sequence
  Heavy chain coupled to scFv region_variant
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGKGGGGSGGGGSGGGGSGGGGSEVQLVESGGGLVQPGGSLRLSCAASAFTFSESWIHWVRQAPGKGLEWVAWISPYGGSTYYADSVKGRFTISADTSKNTAYLQMNSLRAEDTAVYYCARRHWPGGFDYWGQGTLVTVSAGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 82 - protein, 713 aa, Artificial sequence
  Heavy chain coupled to scFv region_variant
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGKGGGGSGGGGSGGGGSGGGGSEVQLVESGGGLVQPGGSLRLSCAASGFSFSDSWIHWVRQAPGKGLEWVAWISPYGGSTYYADTVKGRFTISADTSKNTAYLQMNSLRAEDTAVYYCARRHWPGGFDYWGQGTLVTVSAGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 83 - protein, 713 aa, Artificial sequence
  Heavy chain coupled to scFv region_variant
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGKGGGGSGGGGSGGGGSGGGGSEVQLVESGGGLVQPGGSLRLSCAASGFTFSDTWIHWVRQAPGKGLEWVAWISPYGGTTYYADSVKGRFTISADTSKNTAYLQMNSLRAEDTAVYYCARRHWPGGFDYWGQGTLVTVSAGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR